(19) United States
(12) Patent Application Publication
(10) Pub. No.: US 2006/0134604 A1
(43) Pub. Date: Jun. 22, 2006

(54) Flexible method and apparatus for high throughput production and purification of multiple proteins

(76) Inventors: Mark L. Smith, Davis, CA (US); Kenneth E. Palmer, Vacaville, CA (US); Gregory P. Pogue, Vacaville, CA (US); John A. Lindbo, Vacaville, CA (US); Kathleen M. Hanley, Vacaville, CA (US); David P. Mannion, Davis, CA (US); Gershon M. Wolfe, Davis, CA (US)

Correspondence Address: LARGESCALE BIOLOGY CORPORATION, Roundabout Plaza, Suite 500, 1600 Division Street, Nashville, TN, 37203, US

(21) Appl. No.: 11/303,548

(22) Filed: Dec. 16, 2005

Related U.S. Application Data
(63) Continuation of application No. 10/309,756, filed on Dec. 4, 2002, now abandoned.
(60) Provisional application No. 60/338,725, filed on Dec. 5, 2001.

(51) Int. Cl. C12Q 1/00; G01N 33/50

(52) U.S. Cl. 435/4

(57) ABSTRACT

A plurality of proteins of interest, or peptides of interest, or other genetically expressed materials, are screened and subsequently produced using any of a variety of expression systems. The plurality of proteins are extracted from a plurality of separate, processed green juices, each green juice containing one of the proteins of interest. A multi-channel apparatus processes the various green juices, one green juice per channel. The apparatus is computer controlled such that the various valves in each channel and pump are controlled in an automated manner to extract each protein of interest and deliver each protein of interest into its own storage vessel.

### PRIOR APPLICATION

This application claims priority to U.S. provisional application 60/338,725, filed Dec. 5, 2001, which is incorporated herein by reference in its entirety.

### BACKGROUND OF THE INVENTION

1. Field of the Invention

The present invention relates to flexible high-throughput methods and apparatus for expressing, extracting and purifying relatively large quantities of predetermined recombinant proteins. The invention further relates to a method and apparatus for purifying a plurality of predetermined proteins simultaneously in separate but parallel operating apparatuses. The invention further relates to a method and apparatus for tracking, planning and maintaining a production system for producing a plurality of predetermined proteins simultaneously. The invention further relates to production and purification of a plurality of proteins for use in personalized medicine. The invention further relates to flexible production and purification of a plurality of proteins for use in microarrays. The invention further relates to production and purification of a plurality of proteins for use in protein related research.

2. Background of the Related Art

The study and use of proteins has gained prominence in both the scientific and medical communities in the last few decades as both physicians and researchers recognize the important role proteins play in the physiological and metabolic functions within organisms, such as human beings. Many aspects of proteins are continually being studied, such as protein-protein interactions, glycosylations, identification of protein disease related markers and other characteristics. Proteins are being used in microarrays for use in both research and clinical applications, and large quantities of proteins are required for the production and characterization of antibodies. Hence, the production of proteins is becoming critical for further development in these areas.

There are many protein production techniques, each having its own advantages and limitations. Such methods for producing full-length or partial length proteins include: bacterial based systems, yeast based systems, fungi based systems, insect based systems, mammalian systems and plant systems, such as the GENEWARE® system developed by Large Scale Biology Corporation in Vacaville, Calif.

For all protein systems expressing heterologous proteins, cDNA or DNA sequences of interest are first cloned into a suitable vector which is capable of being transcribed or induced in the host species transformed with the vector DNA. For example, bacterial based systems utilize plasmid, phage or viral-derived vectors for expression of heterologous proteins. Vector DNA containing the nucleic acid sequence of interest (insert DNA) is inserted into the bacteria through standard transformation techniques, including calcium phosphate and electroporation transformation. In addition, many kits are available for the insertion of isolated and purified insert DNA into the selected vector system, making bacterial systems the most widely used for routine expression and purification of heterologous proteins. Although bacterial based systems are frequently used to express heterologous proteins in relatively large quantities, problems of proper folding and lack of post-translational processing may produce functionally inactive molecules. Traditionally, bacterial based systems, therefore, are suitable for only a small range of proteins.

Insect based, and to a lesser extent yeast-based, systems may permit folding, post-translational modification and oligomerization similar to that seen of the native heterologous protein, but fall short of the complexity exhibited by native proteins. An example of an insect based system for producing proteins is the use of baculovirus in insect cells. Plasmid-based Drosophila cell systems are also available, which obviate the necessity for the manipulation and maintenance of baculovirus. Both baculoviral and plasmid-based Drosophila systems utilize vectors, similar to bacterial based systems, for insertion and subsequent expression of heterologous proteins in the host cell. Yeast systems also utilize DNA vectors, such as commercially available pESC, pYES, pNMT, pYD, pPIC and pGAP.

Mammalian expression systems, such as mammalian cell cultures (e.g. NIH 3T3, HeLa, K562, 293 and other cell cultures) transfected with plasmid or phage-based vectors or infected with viral vectors, are capable of substantial post-translational modification. Examples of commercially-available vectors used in mammalian expression systems include viruses, such as adeno associated virus, pFB retroviral vectors and adenovirus, plasmids, such as pACT, pBIND, pCAT, pCI, phRG-CMV, phRG-TK, phRL-TK, pSI and pERV, and phage-based vectors, including pBK, pBK-CMV and pBK-RSV. Mammalian cells, however, may be more problematic to expand to larger scale capabilities because of the culture-intensive work required for expressing foreign proteins. In addition, technical expertise may be required for producing enough cells with the desired quantity of protein. For example, mammalian cells, in particular, may require stable transformation and chromosomal integration of vector DNA because of the inefficiency of transient transfections.

Proteins expressed in plant-based systems also require vectors for the expression of heterologous proteins. For example, Donson et al, U.S. Pat. No. 5,316,931 and U.S. Pat. No. 5,589,367, herein incorporated by reference, demonstrate plant viral vectors suitable for the systemic expression of foreign genetic material in plants. Donson et al. describe plant viral vectors having heterologous subgenomic promoters for the systemic expression of foreign genes. The availability of such recombinant plant viral vectors makes it feasible to produce proteins and peptides of interest recombinantly in plant hosts.

Isolation of proteins produced in bacteria, yeast, insect (baculovirus) and mammalian cultures is also well known. For instance, Qiagen, Valencia Calif., markets materials such as metal affinity resins and magnetic beads compatible with 96-well plate formats for the purification of 6×His-tagged proteins. Such purification techniques are described in A Handbook For High Level Expression And Purification Of 6×His-tagged Proteins published by Qiagen March 2001, and further disclosed in the US Patent Numbers: U.S. Pat. Nos. 4,877,830, 5,047,513, 5,284,933 and 5,310,663, all of which are incorporated herein by reference. However, many of the methods disclosed in the above group of patents and the materials sold by Qiagen, are optimized for isolating quantities of proteins measured in μg or less (not mg quantities) and are further not specifically designed for purification of proteins produced in plants.

Some processes for isolating proteins, peptides and viruses from plants have been described in the literature (Johal, U.S. Pat. No. 4,400,471, Johal, U.S. Pat. No. 4,334,024, Wildman et al., U.S. Pat. No. 4,268,632, Wildman et al., U.S. Pat. No. 4,289,147, Wildman et al., U.S. Pat. No. 4,347,324, Hollo et al., U.S. Pat. No. 3,637,396, Koch, U.S. Pat. No. 4,233,210, and Koch, U.S. Pat. No. 4,250,197, the disclosures of which are herein incorporated by reference in their entirety).

Methodologies have been developed for the cost-effective and large-scale purification of bioactive species produced in plants. These bioactive species may be proteins or peptides, especially recombinant proteins or peptides, or virus particles, especially genetically engineered viruses. Specifically, U.S. Pat. No. 6,037,456 to Garger et al., discloses methods for isolation and purification of large quantities of a protein extracted from, for instance, tobacco plants that have been infected with a recombinant tobacco mosaic virus. The methods disclosed in U.S. Pat. No. 6,037,456 are generally intended for isolation and purification of proteins from large quantities of tobacco plant or other acceptable plant material, where the quantity of protein isolated may be measured in hundreds of grams to kilograms. Further, co-pending and commonly assigned patent application “Flexible Processing Apparatus for Isolating and Purifying Viruses, Soluble Proteins and Peptides from Plant Sources” application Ser. No. 09/970,150 filed Oct. 3, 2001, discloses an automated apparatus for purification of large quantities of proteins produced in plants, again where the quantity of proteins isolated are measurable in hundreds of grams to kilograms. Although the methods described in the patent and pending patent application have many advantages, they are meant for large scale production of material and are not easily applicable to isolation and purification of smaller, more modestly sized quantities of a plurality of proteins, where the quantity of each individual protein is measured in micro-grams to milligrams. U.S. Pat. No. 6,037,456 and co-pending and commonly assigned patent application “Flexible Processing Apparatus for Isolating and Purifying Viruses, Soluble Proteins and Peptides from Plant Sources” application Ser. No. 09/970,150 filed Oct. 3, 2001, are both incorporated herein by reference in their entirely.

There is a need for a flexible system for production and purification of multiple proteins where the proteins may be produced in any of a variety of cultures, and where the proteins are purified in a reliable manner and provide desired quantities of each protein. There is also a need for methods and apparatuses that efficiently perform the production and isolation of 100's μg to several mg of recombinant protein from plant material where the starting biomass ranges from 10 g to less than 10 kg. There is also a need for methods and apparatuses that efficiently perform the production and isolation of similar quantities of recombinant protein produced by bacteria, insect, mammalian and/or yeast cultures. There is also a need for methods and apparatuses that may efficiently perform the production and isolation of proteins associated with proteins of interest to determine proteome structure and relationships within a defined cell, tissue or host organism.

Further, advances in human genome research are opening the door to a new paradigm for practicing medicine that promises to transform healthcare. Personalized medicine, the use of marker-assisted diagnosis and targeted therapies derived from an individual's molecular profile, may impact the way drugs are developed and medicine is practiced. The traditional linear process of drug discovery and development may soon be replaced by an integrated and heuristic approach. Current practice among pharmaceutical manufacturers is to produce massive amounts of a single pharmaceutical, with statistical evidence demonstrating that the pharmaceutical product of interest will only be able to treat a portion of the patient population due to undesirable and adverse reactions in the remaining portions of the target patients. There is a need for production of pharmaceutical products on a small scale where medicines are produced that are tailored to a specific individual or patient population.

Where the virus or protein isolated is intended for production as a pharmaceutical product, consistent and verifiable methodology is required. Therefore, there is a need for automated methodology and apparatus for isolating proteins where the automated apparatus monitors and provides tracking and verification of methodology used in the isolation process.

### SUMMARY OF THE INVENTION

The invention relates to a multiple channel apparatus for parallel and simultaneous purification of a plurality of separate proteins.

The present invention also relates to method and apparatus for simultaneous production and purification of a plurality of proteins.

### BRIEF DESCRIPTION OF THE DRAWINGS

FIG. 1 is a flowchart showing generalized steps of a flexible method for production and purification of a predetermined protein or proteins in accordance with the present invention;

FIG. 2 is a schematic representation of a portion of a computer system employed in one embodiment of the present invention, the depicted computer system assisting in selection of proteins and identification of genetic sequences that express the selected protein or proteins;

FIG. 3 is a flowchart depicting steps of a method for purification of produced protein or proteins in accordance with the present invention;

FIG. 4 is another flowchart depicting subsequent steps of the purification method depicted in FIG. 3 in accordance with the present invention;

FIG. 5 is a schematic diagram representing components of an apparatus for purification of a single protein in accordance with the present invention;

FIG. 6 is a schematic diagram representing components of an alternate embodiment of an apparatus for purification of a single protein in accordance with the present invention;

FIG. 7 is a schematic diagram showing a plurality of apparatuses, such as the depicted in FIG. 5, where the apparatuses operate in parallel for simultaneous purification of a plurality of proteins in accordance with the present invention;

FIG. 8 is a schematic diagram showing an operational step of the apparatus depicted in FIG. 5, with an equilibration solution being passed through the apparatus in accordance with the present invention;

FIG. 9 is a schematic diagram similar to FIG. 8 showing another operational step wherein green juice is being passed through the apparatus in order to capture a protein of interest in a column of the apparatus in accordance with the present invention;

FIG. 10 is a schematic diagram similar to FIGS. 8 and 9, showing another operational step wherein a wash solution is being passed through the apparatus in order to rinse none desirable materials from the column in accordance with the present invention;

FIG. 11 is a schematic diagram similar to FIGS. 8, 9 and 10, showing an eluting solution being passed through the column in order to remove the protein of interest from the apparatus in accordance with the present invention; and

FIG. 12 is a schematic representation of another portion of the computer system employed in one embodiment of the present invention, the depicted portion of the computer system controlling the purification apparatus in accordance with the present invention.

FIG. 13 is a schematic diagram showing the pre-screening for correct transcription of a plurality of vectors using in vitro transcription and gel electrophoresis analysis. Vectors expressing the correct size transcript upon gel electrophoresis are used in further studies to determine the optimal vector and system for protein purification.

FIG. 14 is a schematic diagram showing the pre-screening for correct translation and expression of a plurality of vectors using intact plants and/or cell culture protoplasts systems.

### DETAILED DESCRIPTION OF THE INVENTION

Definitions

In order to provide a clear and consistent understanding of the specification and the claims, including the scope given herein to such terms, the following definitions are provided:

GENEWARE® is a technology developed by Large Scale Biology Corporation, located in Vacaville Calif., to test the function of novel genes and proteins they encode, and to manufacture complex proteins in bulk. GENEWARE® includes the use of a vector modified from a virus to place any gene or a large number of genes within a test organism. The organism then manufactures the gene's protein product, which can be studied, collected and purified.

Preferably, GENEWARE® utilizes tobacco plants or related Nicotiana species infected with a transgenic tobacco mosaic virus. GENEWARE® technology typically includes the use of tobacco plants because the quick-growing tobacco plant provides an extremely useful model organism for studying plant genes, as well as a high-yield factory for manufacturing any protein, either animal or plant, in bulk. A variety of aspects of the GENEWARE® technology are disclosed in the following US Patents commonly assigned to Large Scale Biology Corporation, which are incorporated herein by reference in their entirety: U.S. Pat. No. 5,316,931 to Donson et al., U.S. Pat. No. 5,589,367 to Donson et al., U.S. Pat. No. 5,766,885 to Carrington et al., U.S. Pat. No. 5,811,653 to Turpen, U.S. Pat. No. 5,866,785 to Donson et al., U.S. Pat. No. 5,889,190 to Donson et al., U.S. Pat. No. 5,889,191 to Turpen, U.S. Pat. No. 5,922,602 to Kumagai et al., U.S. Pat. No. 5,965,794 to Turpen, and U.S. Pat. No. 6,054,566 to Donson et al. However, it should be understood that the GENEWARE® technology is applicable to use with plants other than tobacco, such as corn, rice, etc.

In the following description, the terms “bio-mass”, “bio-matter” and “plant source” all refer to any harvested plant, seed or portion of a plant that may be processed to extract or isolate material of interest such as viruses, proteins and/or peptides therefrom. For instance, the bio-matter process may include many types of plants or portions of plants such as seeds, flowers, stalks, stems, roots, tuber, as well as leaf portions of plant material. Typically, the succulent leaves of tobacco plants are ideal for large scale production of predetermined proteins using GENEWARE® technology, but it should be understood from the following description that plants other than tobacco may be used for the production of proteins using GENEWARE® technology.

Alternatively, other plants such as corn, rice, grains or other desirable plants may be utilized for the production of proteins and peptides of interest.

In the following description, the terms “bio-mass” and “bio-matter” may also refer to biological material produced by bacterial based systems, insect based systems, mammalian systems and yeast systems, where the biological material is harvested for the purpose of purifying proteins produced therein in accordance with the methodologies set forth in the description below.

The term “green juice” refers to liquid extracted from processed bio-matter. However, it should be understood that the term green juice may refer to any liquid extracted from a plant material or bio-matter regardless of the extracted liquid's color. For instance, where a protein or proteins is produced using Large Scale Biology Corporation's GENEWARE® technology, the green juice may indeed be green where the green juice originated from bio-matter such as harvested tobacco. However, where proteins of interest are expressed by bacterial based systems, insect based systems, mammalian systems, fungi systems and yeast systems, the liquid extracted therefrom may not have a green color, but in the description below may still be referred to as green juice.

A “virus” is defined herein to include the group consisting of: a virion wherein the virion includes an infectious nucleic acid sequence in combination with one or more viral structural proteins; a non-infectious virion wherein the non-infectious virion includes a non-infectious nucleic acid in combination with one or more viral structural proteins; and aggregates of viral structural proteins wherein there is no nucleic acid sequence present or in combination with the aggregate and wherein the aggregate may include virus-like particles (VLPs). The viruses may be either naturally occurring or derived from recombinant nucleic acid techniques and include any viral-derived nucleic acids that can be adopted whether by design or selection, for replication in whole plants, plant tissues or plant cells.

A “virus population” is defined herein to include one or more viruses as defined above wherein the virus population consists of a homogenous selection of viruses or wherein the virus population consists of a heterogenous selection including any combination and proportion of the viruses.

“Virus-like particles” (VLPs) are defined herein as self-assembling structural proteins wherein the structural proteins are encoded by one or more nucleic acid sequences wherein the nucleic acid sequence(s) is inserted into the genome of a host viral vector.

“Protein and peptides” are defined as being either naturally-occurring proteins and peptides or recombinant proteins and peptides produced via transfection or transgenic transformation.

The terms “protein of interest” “material of interest” and “materials of interest” refer to any material, compound, organic structure or combination of materials to be isolated using the purification methods and/or apparatus in accordance with the present invention. The protein, material or materials of interest may include, but are not limited to: virons, virus-like particles, viruses, proteins and/or peptides, receptors, receptor antagonists, antibodies, single-chain antibodies, enzymes, neuropolypeptides, insulin, antigens, vaccines, peptide hormones, calcitonin, and human growth hormone. Further, the protein, material or materials of interest may be an antimicrobial peptide or protein consisting of protegrins, magainins, cecropins, melittins, indolicidins, defensins, 13defensins, cryptdins, clavainins, plant defensins, nicin and bactenecins.

A “bacteria” is defined herein to include the group consisting of small, unicellular microorganisms that multiply by cell division and whose cell is typically contained within a cell wall, occurring in spherical, rodlike, spiral, or curving shapes and found in virtually all environments.

A “bacterial culture” is herein defined as the maintenance and reproduction of a bacterial population in vitro. The bacterial population is typically clonal in origin, i.e. derives from a single bacterial cell. Therefore, all bacteria within a given bacterial culture should contain the same genetic complement, and in the case of protein expression systems, express the same heterologous protein sequence. The bacterial culture, however, may, in certain circumstances, originate from more than one bacterial cell, and therefore contain a plurality of bacterial cells with differing genetic complements.

A “mammalian cell” is herein defined to include the group consisting of cells derived from a mammalian origin. Sources of mammalian cells include, but are not limited to, tissue, fluids, blood, organs or other biological sources from humans and other mammals.

A “mammalian cell culture” is herein defined to include the group of cells derived from a mammalian source capable of surviving ex-vivo in a cell culture medium. The mammalian cell may be a primary cell, directly derived from a mammalian cell source. More typically, the mammalian cell in a mammalian cell culture will be immortalized, i.e. capable of growth and division through an indeterminate number of passages or divisions.

A “yeast cell” is herein defined to include the group consisting of small, unicellular organisms capable of growth and reproduction through budding or direct division (fission), or by growth as simple irregular filaments (mycelium). The yeast cell may be transformed or transfected with a heterologous vector for expression of a nucleic acid sequence inserted into the heterologous vector. An example of a yeast cell includes Saccharomyces cerevisiae, commonly used for transfection and expression of heterologous proteins.

An “insect cell” is herein defined to include the group of cells derived from an insect source capable of surviving ex-vivo from an insect host. The insect cell may be transformed, transfected or infected with a heterologous vector for expressions of a protein sequence inserted into the heterologous vector. Examples of insect cells include High Five™ cells, Aedes albopictus cells, Drosophila melanogaster cells and Mamestra brassicae cells.

An “affinity tag” is a molecule, ligand or polypeptide attached to a protein (polypeptide) of interest. Examples of affinity tags include, but are not limited to, hexahistidine, other metal tags, streptavidin, biotin, specific epitope markers for antibody purification, glutathione-S-transferase, β-galactosidase, β-amylase and other protein or small molecule tags which may assist in the isolation and purification of expressed proteins.

An “affinity matrix” is a solid-state material bound to a substrate or ligand, which in turn binds selectively to an affinity tag attached to a protein of interest. Upon binding of the affinity tag to the affinity matrix, the protein of interest is retained within the column or other purifying apparatus, and may thus be separated from any impurities present in the green juice. After washing of the affinity matrix, the protein of interest, with the affinity tag attached, may be eluted from the column or other apparatus in a substantially purified form. Examples of affinity matrices include chromatography medium, such as agarose, cellulose, Sepharose, Sephadex and other chromatography medium, polystyrene beads, magnetic beads, filters, membranes and other solid-state materials bound to ligands or substrates which bind to the affinity tag of choice.

A “histidine-tagged protein” is a protein of interest whereby a histidine affinity tag is attached either at the carboxy-terminus, amino terminus or internal to the protein of interest. Typically, the histidine tag consists of six histidine moieties, but may consist of any combination or numerical designation of histidine moieties. The histidine-tagged protein is purified by binding the histidine-tagged protein to a metal affinity matrix, such as Ni-NTA Agarose (manufactured by QIAGEN, Inc.), and washing impurities from the bound affinity matrix. The histidine-tagged protein can then be eluted from the column using acid pH buffering conditions, competitive elution by imidazole or by stripping the metal from the affinity matrix using EDTA (ethylene diamine tetra-acetate).

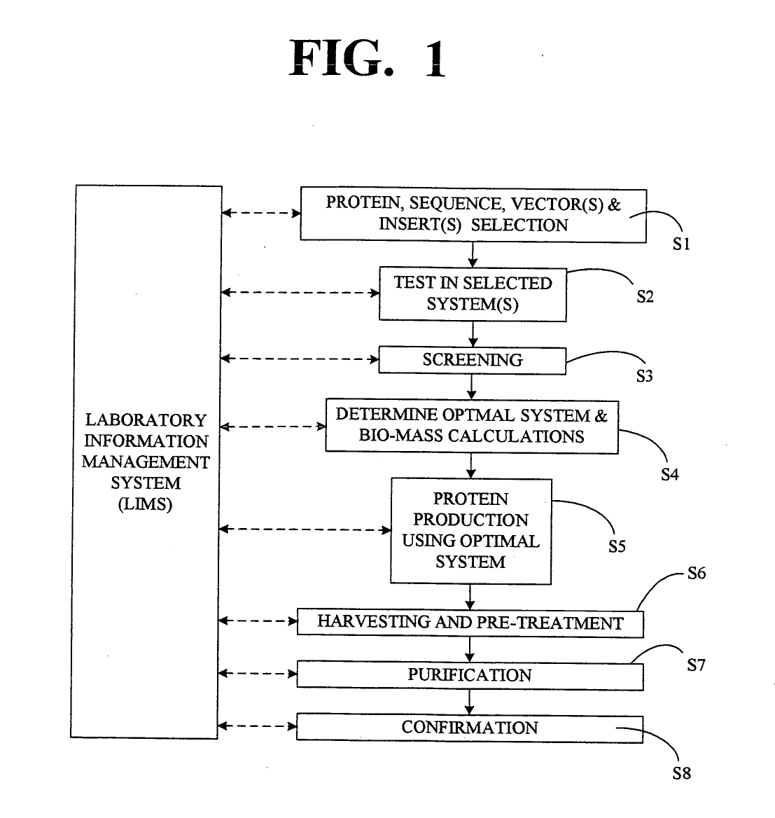

Overview (FIG. 1)

In accordance with the present invention, a protein or proteins of interest are produced by any of a variety of methods, as indicated in FIG. 1. In accordance with one aspect of the present invention, specific quantities of a protein or proteins of interest are produced and purified in an automated manner in order to minimize utilization of materials and time, and to maximize production of the proteins of interest.

FIG. 1 is described in brief in the paragraphs that immediately follow. A more detailed description of appropriate portions of the steps represented in FIG. 1 are included thereafter.

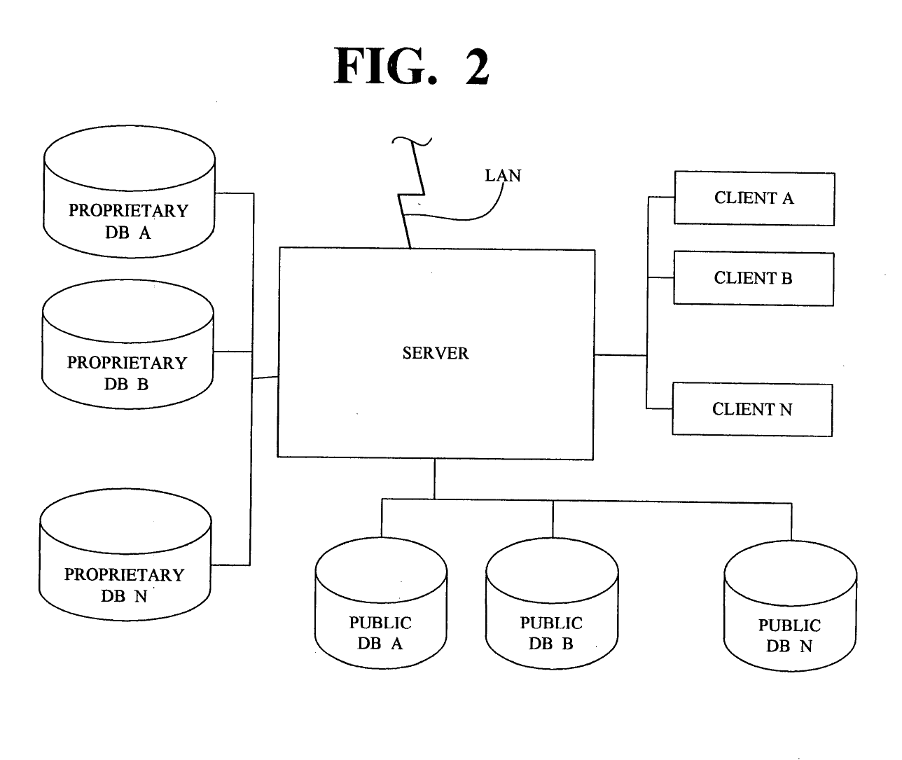

In a first step, shown at box S1 in FIG. 1, a protein or proteins of interest are selected and suitable corresponding vectors & inserts are identified. The protein, vector & insert selection process is described further herein below with respect to FIG. 2.

In the description below, production and purification of at least one protein is described in detail. For most of the following description, production and purification of only one protein is included in order to simplify the description, eliminate redundant language and make this description easier to follow. However, it should be understood from the following description that a plurality of proteins are produced and purified simultaneously in accordance with the present invention.

At box S2 in FIG. 1, after a protein, vector and insert are selected, a suitable organism or system is selected for testing the production of the protein of interest. Specifically, any one or more of the following systems may be utilized, for instance: bacterial based systems, insect based systems, mammalian systems, plant based systems, fungi based systems and yeast systems.

At box S3 in FIG. 1, the protein produced as a result of the test at box S2 in FIG. 1, is screened in order to determine whether or not the protein of interest was properly expressed using the organism or system utilized in the production test. Further, the amount of protein expressed versus the amount of bio-matter produced is also determined. The amount of protein expressed at this stage is relatively small, wherein the protein expressed is screened using a variety of functional and structural tests. The screening process represented at box S3 is thus made up of a number of sub-steps and will be described in greater detail hereinbelow.

A determination is made at box S4 in FIG. 1 with respect to which system (bacterial, insect, mammalian, plant, fungi or yeast) is optimal for expression of the protein of interest. For instance, the GENEWARE® technology is typically used first in a test at box S2. If the protein of interest is not adequately expressed, another organism is tested, such as a bacterial based system, and screened as indicated in box S3. Once an adequate system has been established for the production of the protein of interest, the amount of bio-matter necessary to produce the desired amount of purified protein is calculated, as is described in greater detail below hereinafter. Alternatively, different protein expression systems may also be tested in parallel to determine the optimal system for larger scale expression and purification purposes, i.e. testing bacterial, plant and insect systems simultaneously.

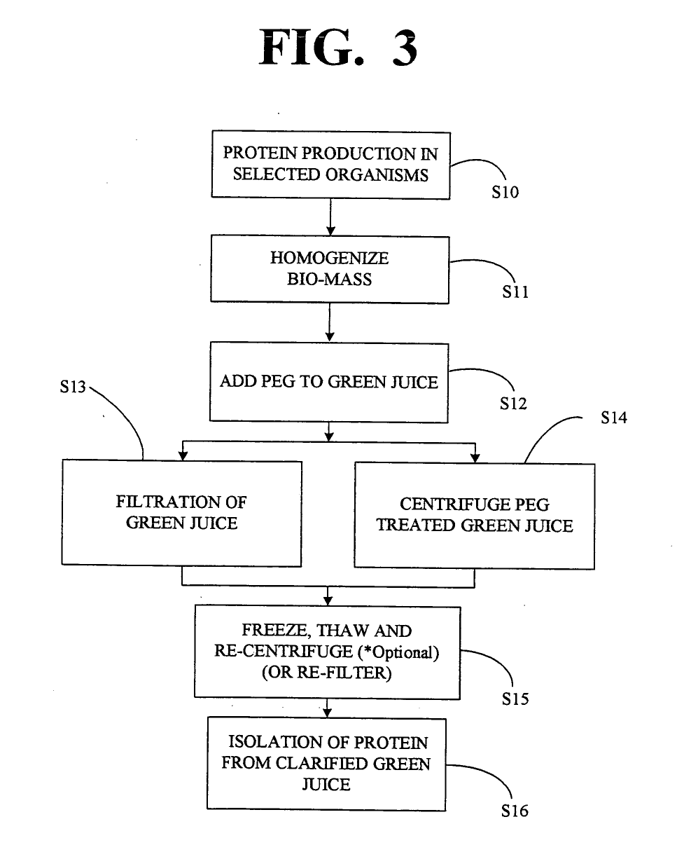

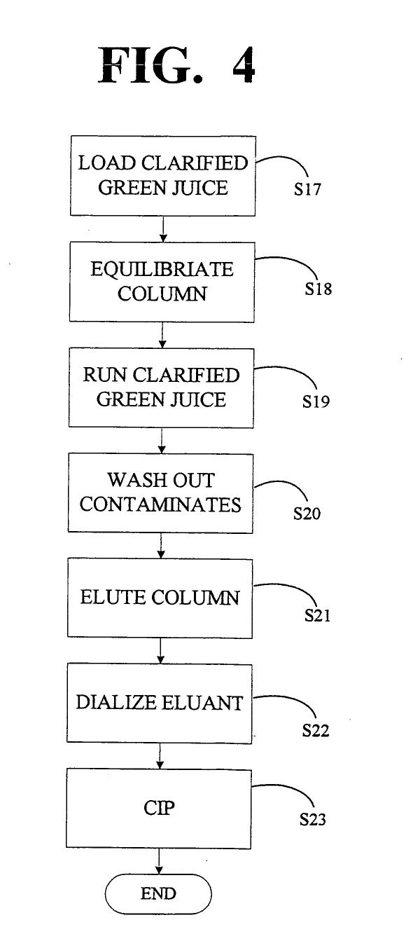

As represented at box S5 in FIG. 1, the protein of interest is then expressed using the determined optimal system or organism. As represented at box S6 in FIG. 1, bio-mass produced by the optimal system is harvested and processed or pre-treated prior to purification, as depicted at box S7. The protein expression, harvesting and pre-treatment steps are described in greater detail below with respect to FIG. 3. The purification steps represented at box S7 in FIG. 1 are described in greater detail below with respect to FIG. 4.

After purification, the purified protein of interest is tested to confirm characteristics and consistency, as represented at box S8 in FIG. 1.

Protein & Insert Selection (FIG. 2)

There are a variety of processes through which protein or proteins of interest may be selected for production and purification, dependent upon the function or purpose thereof. The protein or proteins of interest may be patient specific medicines such as vaccines as described in co-pending U.S. patent application Ser. No. 09/522,900, filed Mar. 10, 2000, where a patient's own DNA provides a sequence for expression of a specific protein. The proteins of interest may alternatively be target proteins for use in, for instance, microarrays or so called protein chips. Protein targets may be chosen to allow evaluation of physiological parameters from collected specimens (blood, serum, urine, sputum, cerebrospinal fluid or any other biological sample), organ function or dysfunction as well as identification of various pathological infectious states.

Where a sequence is needed to express a specific or known protein (for instance, a sequence that is not specifically taken from a patient), the required sequence may be isolated from various databases, both public and proprietary, using a computer system such as that depicted schematically in FIG. 2, where each of these databases may be searched via an in-house client A, B thru N, with access to each of the various databases. Examples of publicly available databases include the National Center for Biotechnology Information (GenBank and BLAST) nucleotide and protein databases, European Molecular Biological Laboratory (SWISS-PROT) nucleotide and protein databases, and other nucleotide and protein databases as well as the medical literature. Examples of proprietary databases include the Human Protein Index (HPI), MEDS (Molecular Effects Of Drugs), MAP (Molecular Anatomy and Pathology) and others unique to many research labs. These sources contain information detailing protein or organism constituents, or may contain information comparing protein expression in diseased versus non-diseased subjects, or normal versus abnormal subjects.

Gene sourcing, or the isolation of nucleic acids of interest, may be produced by a variety of methods, including polymerase chain reaction (PCR), reverse-transcriptase polymerase chain reaction (RT-PCR), colony screening and nucleic acid synthesis. The databases and literature mentioned above, in addition to allowing a researcher or clinician to select proteins expressed for a given state, also contain nucleotide and protein sequence information, allowing suitable target probes to be designed to isolate target cDNA's and proteins of interest.

Considerations of probe design and reaction conditions for isolation are important for isolating specific proteins of interest, known or unknown. Probes to isolate cDNA's of interest may be designed according to protein or DNA sequence information provided by the databases and literature mentioned above. Alternatively, tryptic peptide information from previously unknown proteins isolated on 2-D gels or other methods of protein fractionation and isolation may also be used in probe design. The probes may be synthesized using standard phosphoramidite chemistry, or other nucleic acid synthesis chemistry, incorporating standard deoxynucleotide compounds (dATP, dGTP, dCTP, dGTP), or alternatively may use modified nucleotides that are capable of hybridizing with two or more different deoxynucleotides (dITP or other modified nucleotides). If protein sequences are used as templates for nucleic acid probes, probe sets may consist of at least one pair of primers coding for one permutation of nucleic acid sequence. Alternatively, due to the degeneracy of the amino acid code, more than one pair of primers coding for alternative permutations of the corresponding nucleic acid sequence may be employed. For example, lysine is encoded by two different codon sequences: AAA and AAG. Therefore, a sequence incorporating the amino acid lysine would include both variations within a probe at the lysine position. In addition to synthesizing probes, nucleic acid fragments excised from larger nucleic acid sequences (e.g. cloning vector fragments and other nucleic acid fragments) may also be employed as probes in target nucleic acid isolation.

Probes may also be designed that are similar, but not identical to, known protein sequences. These probes may isolate related proteins that may differ in amino acid sequence composition between individuals, and therefore isolation of such proteins may be difficult using standard probe design techniques. Alternatively, DNA may be screened with nucleic acid probes using decreased stringency conditions, which would allow for the isolation and purification of related, but not identical, DNA sequences. The nucleic acid probes may be used in RT-PCR isolation and cloning from mRNA or total RNA samples. The nucleic acid probes may also be used in genomic DNA cloning from total genomic DNA using PCR amplification or other isolation methodology. Total RNA or genomic DNA may be isolated from animal, plant or bacterial/microbial cells or tissue using standard RNA or DNA purification techniques, e.g. detergent or alkaline lysis, guanidium isothiocyanate, CsCl gradients, Phenol/SDS, Phenol/Chloroform, glass- or silica-based chromatography or other methods, including readily available commercial kits from a variety of manufacturers. Total RNA may be further fractionated on oligo-dT columns or resins to yield poly-A containing mRNA. In addition, mRNA may be directly isolated from cell culture or tissue lysates using standard lysis protocols (alkaline lysis, detergent lysis, mechanical disruption and other lysis methodologies) combined with oligo dT column chromatography.

cDNA strands from reverse transcription of RNA may be copied using DNA polymerase or other available polymerases to yield double-stranded DNA. A variety of standard molecular biology techniques using DNA polymerases may then be used to amplify the double stranded DNA, insert and ligate the amplified cDNA into the appropriate expression vector for further analysis. Alternatively, genomic DNA may be directly PCR amplified using DNA polymerase or other available polymerases. As with amplified cDNA, amplified genomic DNA can be inserted and ligated into the appropriate expression or replication vector for further analysis.

An alternative protocol for isolating a DNA sequence of interest is the synthesis of an insert sequence, and its complementary binding strand, through standard DNA synthesis protocols. For example, complementary DNA strands may be synthesized using standard phosphoramidite chemistry. For cloning purposes, assymetric restriction enzyme sequences may also be incorporated into the synthesized strand for directional cloning into a replication and/or expression vector. Alternatively, blunt end ligation of restriction enzyme linkers after annealing of the DNA strands may be accomplished using standard molecular biology ligation protocols. Using DNA synthesis methodologies, picogram, nanogram, microgram or milligram quantities, usually dependent upon the length of the sequence, may be synthesized and purified, which may avoid potential amplification artifacts that may be introduced with DNA polymerase enzymes. DNA synthesis may also be combined with PCR amplification to amplify sufficient quantities for DNA insertion and subsequent replication of DNA into the appropriate vector.

Yet another method is the use of colony screening of bacterial hosts containing a plurality of vector inserts. Typically, the vector inserts may comprise a plurality of nucleic acid sequences isolated from a specific host tissue, organ or condition. For example, commercial bacterial “libraries” are available that correspond to a plurality of vector inserts from mouse liver, or mice that are phenotypic for a specific disease. Isolated probes from above may be used to screen a large number of bacterial clones transferred onto a solid medium, such as nitrocellulose or nylon filters or membranes. The bacterial clones on the solid medium are lysed, and the DNA contained within each clone denatured and bound to the medium, so that the pattern of colonies is replaced by an identical pattern of bound DNA. The medium is then hybridized to labeled probes which identify the clone containing the DNA sequence of interest. The clone is isolated, amplified by large scale culture and the DNA isolated and excised for manipulation into other vectors of interest.

Vector Selection

In accordance with the present invention, the isolated DNA sequence of interest is inserted into a vector to allow the production of recombinant proteins of interest by any of a variety of methods, such as bacterial based systems, insect based systems, mammalian systems and yeast systems or by using aspects of GENEWARE® technology, as described above and in the above identified patents commonly assigned with the assignee of the present invention. Specifically, in the GENEWARE® system, a virus is genetically manipulated to include a vector, a tag and the genetic sequence or insert of interest, selected specifically for the protein it encodes. The virus is then applied to leafy plant tissue such as the leaves of a tobacco plant, thereby infecting the organism. The plant and virus work to express the specific protein, and the protein is subsequently extracted from the plant tissue and then purified. This basic workflow of the methodology of the present invention is described in greater detail below along with a detailed description of apparatus used to effect the methodology of the present invention. Further, a computer system is also described for tracking the work flow and assisting in determining various aspects of the process in a manner described more clearly below.

In accordance with the present invention, where the GENEWARE® technology is employed, specific vectors and inserts are selected for insertion into a tobacco mosaic virus or other suitable virus. One insert is selected for the specific protein encoded by the genetic sequence of that insert, as is indicated at S1 in FIG. 1. As will be understood more clearly from the following description, a plurality of viruses may be utilized, one insert per virus, such that a plurality of proteins may be expressed simultaneously. Further, a vector or plurality of vectors is selected from a variety of vectors for each insert for insertion into a virus. For instance, not all vectors will function with every insert. Therefore, a plurality of vectors may be experimented with to test expression of the desired protein.

As mentioned above, a variety of cloning and expression vectors may be employed for use in protein expression and purification, depending upon the host system used. Typically, cloning and expression vectors are only able to transfect, transform or infect one specific host system (e.g. only plants or bacteria). However, there are cloning and expression vectors, by the nature of the nucleic acid sequences contained within, which are capable of transfecting, transforming or infecting a plurality of host systems. Those of ordinary skill in the art will appreciate that vectors may be designed to transfect, transform or infect a variety of host systems, and any vector capable of transfecting, transforming or infecting and subsequently expressing the vector insert nucleic acid sequence within the host is contemplated within the scope of this invention.

As mentioned above, the choice of vector used is dependent upon the host system contemplated in the purification procedure. For example, plant systems may use viral vectors, derived either from RNA or DNA viruses, for the introduction and expression of heterologous protein sequences. RNA viral vectors are preferred for their high expression levels and host ranges. U.S. Pat. No. 5,316,931, which is incorporated in its entirety herein by reference, describes plant viral vectors having heterologous subgenomic promoters which allow systemic infection of plant hosts and stable transcription or expression in the plant host of foreign gene sequences. Similarly, U.S. Pat. No. 5,811,653, which is incorporated herein by reference, describes an RNA viral vector from the tobamovirus group capable of overexpressing genes in tobacco plants. U.S. Pat. No. 5,977,438, which is also incorporated herein by reference, describes an RNA viral vector which fuses foreign genes to RNA viral proteins (e.g. coat protein), producing relatively large amounts of foreign protein in the form of a fusion protein.

A preferred embodiment may be an RNA viral vector from the tobamovirus family. An example of this is found in the tobacco mosaic virus-derived GENEWARE vector. In the GENEWARE vector, the TMV Replicase coding sequence is upstream of the coding sequence for TMV movement protein. A cDNA ORF (open reading frame), which is ligated 3′ of the TMV movement protein, is joined in frame to a hexahistidine affinity tag polypeptide coding sequence or any other affinity tag coding sequence either at the 3′ or 5′ end. The addition of an affinity tag coding sequence within the cloning and expression vector allows the purification of proteins from complex mixtures by binding the affinity tag-protein of interest to an affinity matrix and subsequently washing the same until all impurities are removed. The protein and affinity tag can then be eluted from the affinity matrix in a substantially pure form. The vector may be optimized for higher expression in protoplasts and inoculated leaves, may be cloning friendly with multiple restriction enzyme sites in the polylinker region 5′ of the cDNA insertion site and contain termination sequences for proper termination of the expressed protein. For example, a tobacco mosaic virus-derived vector may include the TMV replicase coding sequence, which may substantially increase expression in both protoplasts and inoculated leaves. In addition, restriction enzyme sites, including EcoRI, BamHI, SmaI, SacI, NotI, XbaI, SpeI, XhoI, Sap I or other restriction enzyme sites may be contained within a multiple cloning site polylinker sequence flanking the insertion site of the desired nucleic acid sequence. Other RNA viral vectors besides tobamovirus vectors may also be employed, including, but not limited to, rice dwarf virus, wound tumor virus, turnip yellow mosaic virus (tymovirus), rice necrosis virus, cucumber mosaic virus (cucumovirus), barley yellow dwarf virus (luterovirus), tobacco ringspot virus (nepovirus), potato virus X (potexvirus), potato virus Y (potyvirus), tobacco necrosis virus, tobacco rattle virus (tobravirus), tomato busy stunt virus (tombusvirus), watermelon mosaic virus, brome mosaic virus (bromovirus) and other RNA viruses. The RNA in single-stranded RNA viruses may be either a plus (+) or a minus (−) strand.

DNA viral vectors may also be employed for subsequent inoculation and protein expression in host plants. Examples of DNA viral vectors include, but is not limited to, caulimoviruses such as Cauliflower mosaic virus, Cassaya latent virus, bean golden mosaic virus, Chloris striate mosaic virus, maize streak viruses and other DNA viruses. Alternatively, Agrobacterium tumefaciens plasmid vectors may also be employed for Ti-mediated plant transformation.

Vectors, as mentioned above, may contain affinity tag sequences (hexahistidine, other metal affinity tags, streptavidin, specific epitope markers for antibody purification, glutathione-S-transferase, β-galactosidase and other tags which may assist in the isolation and purification of expressed proteins) and multiple cloning site linker sequences to assist in the cloning and purification of the protein of interest. DNA or RNA viral vectors may also contain a nucleic acid sequence coding for a signal peptide in order to direct expression of the foreign protein for secretion into interstitial fluid or the culture medium. This may simplify and enhance purification efforts due to the limited amount of endogenous proteins secreted into the interstitial fluid compartment by the plant host. An example of this may include incorporation or ligation of the sequence coding for the rice alpha-amylase signal peptide, which directs secretion of the chimeric protein into the interstitial space of the infected leaf or other plant component transfected.

In addition to plant viral vectors for plant transformation and subsequent expression and purification, mammalian or prokaryotic expression vectors may be employed for subsequent transfection or transformation into a prokaryotic or mammalian host. A preferred embodiment may be a dual mammalian/E. coli expression vector capable of transcription and subsequent expression in both bacterial and mammalian hosts. An example of this is the expression vector MEV (Mammalian Expression Vector), which contains a polylinker site with traditional restriction enzyme cloning sites (BamHI, EcoRI, SmaI, NotI, etc.), as well as SapI/EarI cloning sites. The mammalian CMV immediate-early enhancer promoter unit is located upstream and separated by an intron from the bacterial promoter unit. A Shine-Dalgarno/Kozak sequence is included for efficient expression. A histidine-tag coding sequence for efficient isolation and purification of expressed proteins is also included, which is expressed in E. coli only due to the presence of SupE/F sites.

Vectors may be constructed to allow simultaneous insertion of a nucleic acid insert into a plurality of vectors for testing in different systems. For example, vectors which are capable of expression in mammalian, bacterial and plant systems may contain the same restriction enzyme sites in the linker region of the vector DNA. Thus, a cDNA insert may be cloned into corresponding restriction enzyme sites in several different vectors, such as MEV and GENEWARE vector, simultaneously, ensuring identical frame placement of all vectors for a given cDNA insert.

As with plant viral vectors, it may also be desirable to incorporate affinity tag coding sequences (hexa-histidine, other metal tags, streptavidin, protein A, calmodulin binding protein (CBP), chitin binding domain (CBD), specific epitope markers for antibody purification, and other tags which may assist in the isolation and purification of expressed proteins) and multiple cloning site linker sequences for insertion and purification purposes into other vector DNA. Signal peptide sequences which direct the secretion of the expressed protein for packaging and subsequent secretion into the extracellular fluid matrix or culture medium may also be utilized for simplifying and enhancing purification of the expressed protein. In addition, other gene sequences which enhance the function of the vector package may also be incorporated into the vector sequence. An example of this is the incorporation of the gene sequence encoding granulocyte/macrophage-colony stimulating factor (GM-CSF) into the mammalian expression vector for proteins that may be used in the generation of antibodies or other immune responses (e.g. vaccines). GM-CSF recruits antigen presenting cells (APC; dendritic cells and macrophages), as well as enhances production of stem cell growth factors. This may result in the stimulation of the immunomodulatory system, which may increase the ability of a mammalian host to produce antibodies of higher specificity and affinity.

Affinity tags may also be used to isolate protein complexes bound to the tagged protein of interest. For example, a tandem affinity purification (TAP) tag system, previously demonstrated in yeast (Rigaut et al., 1999 Nature Biotechnology 17, 1030-1032; Gavin et al., 2002 Nature 415, 141-147), may be used to isolate proteomes, whereby the protein of interest contains the TAP tag. In the TAP system, the protein of interest is attached to two affinity markers (e.g. protein A and CBP) separated by a specific TEV protease cleavage sequence. In order to achieve expression of the protein of interest at a natural level in the yeast system, a DNA cassette encoding the TAP tag is integrated by homologous recombination into the genome of a haploid yeast cell in frame with the protein of interest.

The TAP system consists of a two-step purification system to decrease non-specific binding. The affinity purification systems, combined with the presence of the specific TEV protease cleavage sequence, also allow mild elution conditions, increasing the chances of isolating proteomes or protein complexes. Typically, a TAP purification consists first of attaching in frame a TAP gene cassette, containing the coding sequences for two affinity markers separated by the specific TEV protease cleavage sequence, onto the end of a gene sequence of interest. The TAP gene cassette may be attached to the end of a protein coding sequence by PCR cloning and amplification or by insertion and ligation into a suitable vector containing the protein of interest. The TAP-tagged protein coding sequence of interest is then inserted into a host cell, expressed, and proteins associated with the protein of interest isolated and identified. Alternatively, the TAP gene cassette may also be attached in vivo to the protein of interest by homologous recombination in frame within the chromosome of the host organism. The TAP-tagged protein sequence of interest is then expressed in vivo and associated proteins isolated.

Isolation of associated proteins is through a two-step purification procedure. A first affinity purification is performed to initially isolate any proteins associated with the TAP-tagged protein of interest. The proteome or protein complex is eluted from the first affinity purification matrix by cleavage with TEV protease, allowing a mild elution from the affinity matrix. In order to remove any non-specific proteins, contaminants and TEV protease, a second affinity purification is performed using a second affinity purification matrix. The associated proteins are then released from the bound protein of interest using EGTA elution. The isolated proteins are further isolated using denaturing gel electrophoresis. The individual protein bands are digested with trypsin and analyzed by matrix-assisted laser desorption/ionization time-of-flight mass spectrometry (MALDI-TOF MS). The proteins may be identified by known database search algorithms such as Profound™ and Protein Prospector™ against databases such as NCBI SWISS-PROT or other databases known to those of skill in the art, and analyzed for protein content within the proteomes as well as between different isolated proteome structures.

Other mammalian, prokaryotic, insect, fungi or yeast vector may also be used in conjunction with the methods and compositions disclosed herein. These may include, but are not limited to, pBluescript, pCDNA3.1, pHAT, pIRES, pGBKT7, pVPack, pCMV-tag, pDual-GC, pBk-CMV, pIB-E, pMelBac, plueBac4.5/V5-His, pYD1, pPIC9K, pYES2, pIB/V5-His, pIZT/V5-His, pIZ/V5-His, pNMT1, pPICZ, pNMTsl, pMET, pPIC3JK, pGAPZ, pAO815, as well as other vectors which incorporate genetic elements necessary for expression in prokaryotic, mammalian, yeast, fungi or insect cell systems or a combination of genetic elements from different systems which allow expression in at least one of the expression systems above.

Screening of Recombinant Vectors

Transcription Analysis

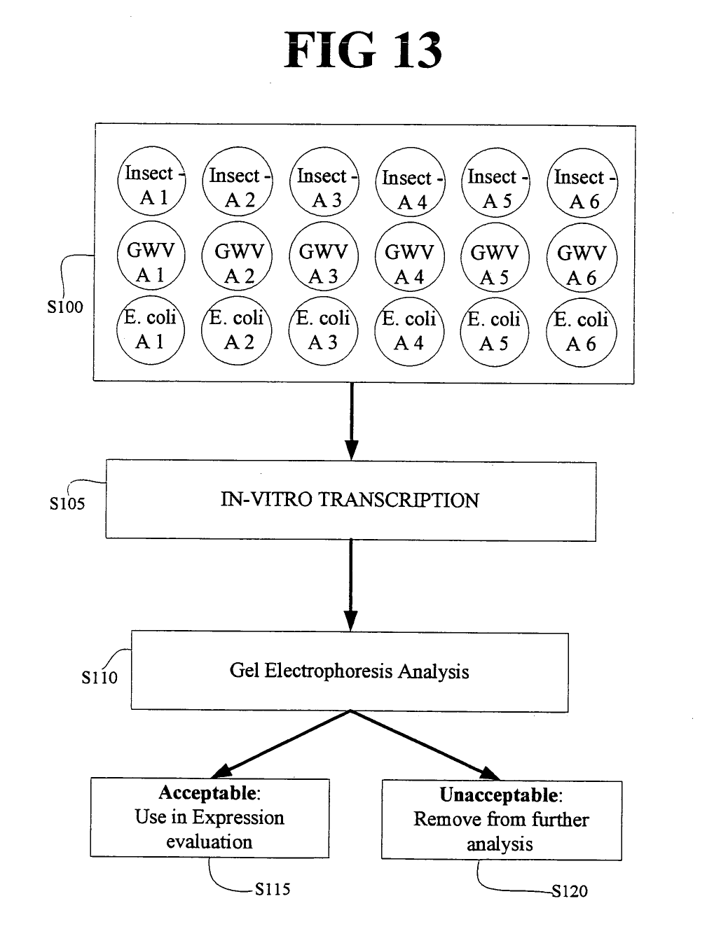

Prior to a scaled-up expression of the protein of interest, vectors containing the sequence of interest may be evaluated for correct transcription of targets. Correct insertion of cDNA's into cloning vectors may be evaluated using an in vitro, prokaryotic, eukaryotic or plant transcription system in an array format, followed by size analysis of transcripts produced. A preferred embodiment is seen in FIG. 13, where in vitro transcription and analysis is used to pre-screen vector constructs that may be used for expression and purification. The vector constructs, previously chosen from cloning of the inserts into the appropriate vectors, may be placed in an array format represented at Step S100, in this example a 3×6 array format, and analyzed simultaneously. The vector constructs may be chosen from a variety of systems, including insect, plant (GWV=Geneware Vector®) and bacterial (E. coli=Escherichia coli). Because insertion of insert DNA into a cloning may be unsuccessful, it may be useful to screen a plurality of clones from a cloning attempt, represented here by A1 through A6. Vector constructs may contain a T7 promoter, or any other promoter capable of in vitro transcription, upstream of the cDNA insert. T7 in vitro transcription, represented by step S105 may be initiated with the addition of bacterial T7 RNA polymerase followed by subsequent analysis at step S110 of the length of transcripts on RNA agarose, polyacrylamide or other type of RNA size separating gel electrophoresis system or RNA analysis system. Successful S115 and unsuccessful S120 reactions are scored according to the estimated size of the transcript, whereby individual (or whole plate) transcription reactions may be repeated if the number of acceptable transcripts falls below a pre-determined threshold, e.g. 50-75%. In cases where the number of acceptable transcripts fall below the pre-determined threshold mentioned above, the clones may be re-transformed into an appropriate host vector for subsequent amplification, repurification of the T7 vector clones and subsequent transcription using T7 RNA polymerase.

Other transcription systems may be used for evaluation of successful transcript production in each cloned cDNA vector. These may include SP6 transcription, T3 RNA polymerase or any other transcription system. For each system, the appropriate promoters (SP6 and T3 promoters, respectively) are necessary for recognition by the RNA polymerase. After addition of the RNA polymerase and transcription, the transcripts may be analyzed by polyacylamide, agarose or other gel electrophoresis for appropriate size transcripts present.

Expression Analysis

After confirmation of correct insertion of the sequence of interest into a vector, evaluation of protein expression may occur to determine the optimal vector and conditions for protein expression. Alternatively, vector constructs may be tested directly for protein expression, bypassing any transcription or RNA analysis. In all formats, evaluation of protein expression at a small scale may be used as a screening methodology to determine the optimal protein expression system for use with the described protein purification methodology.

Evaluation of protein expression may occur in a variety of systems, including plants, bacteria, yeast, fungi, insect and mammalian systems. A preferred system is the use of a plant expression system for expression of the protein of interest. However, as mentioned above, some proteins may not express well or at all in a plant expression system. Other systems may also be convenient for expression purposes, depending upon the type of equipment available for culture and amplification of the host system. Alternative embodiments for testing of the vector and inserts include bacterial, fungi, yeast, insect and mammalian systems.

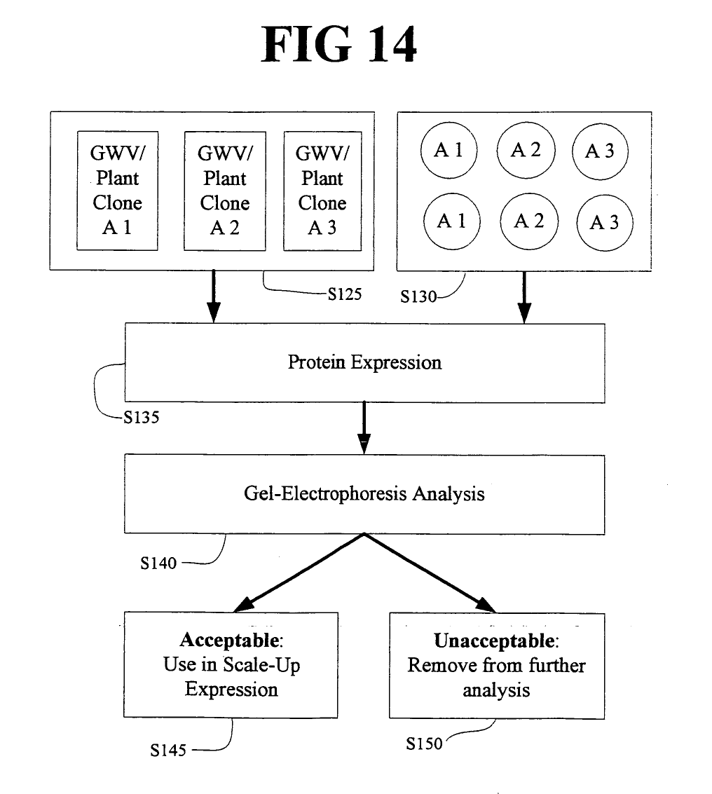

Protein expression in plants may be evaluated in a variety of ways. A preferred embodiment of the invention is to evaluate protein expression in both protoplasts cultures represented at step S130 and intact plants at step S125, as depicted in FIG. 14. Intact plants S125 may be infected with the appropriate viral vector expressing the protein sequence of interest (e.g. GWV=Geneware Vector®). Preferably, young leaves or stalk are infected with encapsidated viral vectors containing the sequence of interest. Viral vectors may also be delivered into the plant host by electroporation, micro projectiles (e.g. small microscopic titanium or gold pellets coated with the recombinant viral vector detonated into the cells at a high velocity) or other methods which introduce heterologous nucleic acid expressing the protein sequence of interest into intact plant cells, as represented at step S135. The plant vectors may be inserted into a small number of organisms, such as one to three tobacco plants that are 17-28 days old (but preferably 21 days old). The plants infected with the virus are allowed to grow for a predetermined length of time, for instance, 10 to 16 days (but more preferably 12 days), as is indicated by S2 in FIG. 1. The plants may be harvested and processed by grinding the infected leaf or stalk, e.g. between twin-roller drums to simultaneously grind and extract homogenate, or any other process which grinds the plant material into fine pieces. The resulting extract, the green juice, is further processed to purify the protein expressed. For example, the green juice may be combined in a 1:2 ratio with 25 mM Tris pH 8.0, 500 mM NaCl, 2 mM PMSF, 7 mM β-mercaptoethanol buffer adjusted to 4% weight per volume PEG, and after half an hour at 4° C., centrifuged to obtain a clarified green juice. The clarified green juice is added to a 96-well MBPP (melt-blown polypropylene) filter plate containing 20 μl of a Ni-NTA bead slurry. The green juice and Ni-NTA beads are incubated for 1 hour at room temperature and spun at 1000×G for 5 minutes to remove green juice from the wells. The Ni-NTA beads are washed to remove non-specifically bound green juice proteins. The affinity tagged proteins of interest are eluted from the beads by either imidazole or EDTA incubation. The eluted protein is spun into a second 96-well plate and then tested for protein presence by SDS-PAGE (sodium dodecyl sulfate polyacrylamide gel electrophoresis). As one skilled in the art recognizes, the purification of the green juice proteins may be any of a variety of processes, such as: the process described in commonly assigned U.S. Pat. No. 6,037,456 to Garger et al.; the process described in The QIAexpressionist™ A Handbook for High-Level Expression and Purification of 6×His-tagged Proteins, published by Qiagen, Valencia, Calif., March 2001. Only small amounts of the expressed protein are likely produced in this initial test, and the amounts may only be measurable in μg's or smaller quantities.

It may be desirable, instead of infecting intact plants, to transfect plant cell cultures, or protoplasts, with the plant vectors for protein expression S130 (FIG. 14). For plant cell culture assays, protoplasts are prepared according to standard molecular biology protocols. Protoplasts may be derived from a wide variety of sources, including leaf, anthers, shoot, root tips or any other plant tissue available. A preferred embodiment is digestion of leafy material from Nicotiana tabacum. Other suitable plants may be utilized, and may be dependent upon the type of vector chosen for propagating nucleic acid or protein expression. This includes Solanum tuberosum, Arabidopsis thaliana, other angiosperms or vascular plants, as well as other types of plants, including mosses and liverworts. Single cell protoplasts suspensions may be generated by first collecting explant tissue, such as leaves, and sterilising tissue surfaces using standard techniques, such as sodium hypochlorite exposure. The explants are then digested with an appropriate cocktail of enzymes to yield single cell suspensions. A preferred embodiment may employ pectinase (e.g. Macerozyme R10, Pectolyase Y23, Rhozyme HP150 and other pectinases) and cellulase (Cellulase, Cellulysin, Driselase and other cellulases), however, other enzyme cocktails which digest interstitial tissue surrounding individual plant cells may be utilized.

Protoplast suspensions may be aliquoted into microtiter plates (in this example, a 2×3 microtiter plate array, but other microtiter plate formats can be utilized) after enzymatic digestion, washing and culture using an appropriate medium, preferably in duplicate. A preferred embodiment may utilize commercially available basal Murashige and Skoog medium, although other medium preparations which provide a balanced mixture of macro and micro-elements, soluble carbon sources, nitrogen vitamins and other growth factors necessary for maintenance of protoplasts in vitro. It is well known to those of ordinary skill in the art that many different combinations and ranges of media constituents can be used successfully for protoplast expansion.

After suspension of protoplasts and subsequent incubation in a suitable medium, protoplast cells may be transfected with the DNA or RNA using a variety of methods. Preferably, the gene of interest is incorporated into a GENEWARE® vector, is packaged or encapsidated and the encapsidated virus is used to infect protoplasts. In this way, protoplasts are transiently transfected with a suitable vector and induced in vitro to express the desired protein. Direct DNA microinjection, electroporation, liposome carriers, particle bombardment (biolistics), silicon carbide fibers or other methods may also be used to introduce and express foreign genes in plant cells.

Alternatively, Agrobacterium tumefaciens-mediated Ti transfer of cloned DNA may also be used to introduce and express foreign genes in plant cells. For example, cloned DNA may be inserted into a suitable vector which is taken up by Agrobacterium. The protoplasts or intact plants are then incubated in the presence of the DNA-containing Agrobacterium. Agrobacterium, through the presence of the Ti gene, mediates the transformation and integration of the insert DNA into the plant cell host. Protein expression may subsequently be induced under the control of inducible promoters co-transfected with the DNA of interest, or natural promoters may be transfected which may subsequently place control of expression under the plant host. Such natural promoters may include constitutively active promoters, which may be modified to express foreign genes at high levels.

Protoplasts may also be used as an initial screening tool for determining if an intracellular or secretory pathway is used for protein expression. Microwell culture plates are first centrifuged to separate protoplast cells from cell culture media. The media is aspirated and collected for parallel purification along with protoplast cell lysate. Both the protoplast cell lysate and media, as well as intact plant homogenate suspensions, are added to separate wells in a 96-well filter plate containing metal binding matrices, such as Ni-NTA beads or Ni-chelating disks (Swell-Gel, Pierce). The flow through fraction is discarded, and the metal binding matrix (such as Ni-agarose) washed with 40 mM Imidazole/0.5 M NaCl/Phosphate buffer (pH 7.9). The bound proteins are then eluted with 1 M Imidazole/0.5 M NaCl/Phosphate buffer (pH 7.9) and analyzed on 1-D or 2-D polyacrylamide gels (step S140 in FIG. 14). The target bands are analyzed if the target protein of appropriate size is produced and the expression level quantified. The target proteins for protoplast samples are also noted for secreted or intracellular protein pathway dependent upon which sample isolate contains His-tagged proteins.

In addition to identification on 1-D gels, target bands may also be excised from the 1-D polyacrylamide gels and analysed by tryptic MALDI-TOF (Matrix Assisted Laser Desorption/Ioniazation-Time of Flight Mass Spectrometry) which may ensure correct insertion of the cDNA into the vector (correct reading frame) and confirm correct protein expression. Tryptic MALDI may be performed by first eluting protein from the polyacrylamide gel, followed by trypsin digestion of the proteins, purification of the fragments, lyophillization and subsequent solubilization in the proper solvent. The sample is then analyzed using MALDI-TOF Mass Spectrometry or any other ion desorption method allowing sequential peptide cleavage and mass measurements. As an alternative embodiment, target bands may be excised from the 1-D polyacrylamide gels or transferred to nitrocellulose or PVDF membranes and eluted from the membranes. The isolated protein band can then be sequenced using standard protein sequencing techniques (e.g. Edman degradation or any other protein sequence method). In addition, trypsin digestion may also be performed on the isolated protein, after which standard protein sequencing techniques are applied (e.g. Edman degradation or any other protein sequence method).

After analysis on 1-D polyacrylamide gels and MALDI-TOF MS, the probability that the protein expressed is the correct protein may be calculated using standard database analysis.

In a manner similar to that described above with respect to FIG. 13, analysis at step S140 is used to determine the acceptability, at step S145, or the unacceptability, at step S150, of expressed proteins, as shown in FIG. 14.

Vectors and their insert may also be evaluated using bacterial, fungi, yeast, insect and mammalian systems. For example, in situations where no protein is expressed from transfected protoplast cultures or infected plants, the corresponding MEV cDNA clone may be analyzed for expression in E. coli to assure that DNA transfection error into plants or protoplasts is not the cause of the lack of protein expression. Alternatively, bacterial, fungi, yeast or insect may be tested in parallel with a plant expression system to determine the optimal system for protein expression and purification.

There are many methods known to one of ordinary skill in the art for expressing foreign proteins from a cDNA vector in a prokaryotic host. A preferred embodiment may include the transformation of a suitable host strain of E. coli, such as NovaBlue DE3, with MEV vector containing cDNA or genomic DNA inserts in a 96-well format. The transformants may be plated on solid media with selective antibiotics, depending on the vector used, and grown overnight in a deep 96-well block at 37° C. After overnight growth, the E. coli cultures may be diluted into fresh media containing isothiopropyl galactoside (IPTG) to induce expression of the protein through the β-galactosidase promoter in the vector. Alternatively, other strains of E. coli or suitable prokaryotic host strain may be used for propagating vector DNA and their inserts, as well as other vectors with alternative inducible promoter systems, such as temperature-dependent expression or other inducible systems.

After logarithmic growth for a defined period of time, 2 microliters of culture may be spotted onto a nitrocellulose membrane in an 8×12 grid, and a Western blot may be performed using antibody to the target protein or tag. Alternatively, the expressed protein, with its attached hexahistidine tag, may be isolated and purified as above on a SwellGel Ni chelating matrix in a 96-well filter plate format. The eluted protein may be analyzed on 1-D polyacrylamide gels for determination of proper size expression. In addition, tryptic MALDI-TOF may be performed on excised protein bands for further identification.

Protein Expression Scale-Up

After protein evaluation and screening, a larger scale protein expression and purification may be commenced. The evaluation of the protein, as is indicated at S3 in FIG. 1, includes: confirmation that the desired protein was expressed; plant mass obtained per plant; and target protein expression level. The plant mass obtained and target protein expression level are then used to calculate the number of organisms (i.e. tobacco plants) necessary to produce a desired amount of the target protein, as is indicated at S4 in FIG. 1. The number of organisms (i.e tobacco plants) necessary to produce the desired amount of protein is planted, as is indicated at S4 in FIG. 1. Further the organisms are infected with the transgenic virus and the protein allowed to express in the organism. It should be understood that a series of steps similar to steps S1 thru S4 are applicable to use of mammalian, yeast, insect or bacteria based protein producing systems.

The steps depicted in FIG. 1 at boxes S6 and S7 are now described in greater detail with reference to FIGS. 3 and 4. As indicated at box S10 in FIG. 3, the proteins are allowed to express in the selected system, such as the GENEWARE® system.

The tobacco plants are then harvested, and disintegrated in, for instance, a Waring blender or commercial juicer to release the desired protein or proteins from the cells of the leaves in the form of green juice, as indicated at S11 in FIG. 3. Typically, a biomass to extraction buffer ratio of 1:2 is employed and the buffer can be vacuum infiltrated into the plant material prior to extraction. The typical extraction composition is 25 mM Tris pH 8.0, 500 mM NaCl, 2 mM PMSF, 7 mM B-mercaptoethanol and may also include up to 1% w/v Tween-20 and up to 5% w/v sodium ascorbate. Next, as indicated at S12 in FIG. 3, the green juice is then treated with a clarifying agent, such as poly-ethylene glycol (PEG), typically 4% w/v in the presence of NaCl (concentration range of 300 mM to 2M). However, it should be understood that clarifying agents such as polyvinylpyrolidone (PVPP) may be employed either alone or in combination with PEG. PEG has been found by the inventors to be a clarifying agent allowing removal of a significant amount of larger chlorophyll-containing protein & membrane complexes, rendering the green juice sufficiently clear to permit loading onto a chromatography column while leaving smaller size proteins (and the protein of interest) in suspension in the green juice. Specifically, when PEG is added to the green juice, which is an aqueous solution, the PEG causes larger proteins to interact and aggregate making them easier to centrifuge or filter out of the solution.

After being treated with a clarifying agent, the green juice may be further processed in one of at least two alternative manners. First, as depicted at S13 in FIG. 3, the PEG treated green juice may be subjected to a filtration process that includes first treating the green juice with a filtration aid, such as perlite (ground volcanic rock), that is mixed in with the green juice at a final concentration ranging from 1% w/v to 10% w/v and preferably 4% w/v. Thereafter the green juice is passed through a glass fiber filter with an average pore size of 1.2 microns, coated with perlite wherein the clarified green juice passes through the filter, but the perlite and larger protein aggregates are retained by the filter. Thereafter the clarified green juice may be subjected to the step described at S15 in FIG. 3. However, it should be understood that the step depicted at S15 in FIG. 3 is an optional step, and may not be required.

Alternatively, after step S12, the PEG treated green juice may be subjected to centrifugation at a force of 3,700 G for approximately 20 minutes in order to separate the larger protein aggregate from the clarified green juice, as indicated at step S14 in FIG. 3. Debris which does not pellet efficiently is subsequently removed by filtration through miracloth. In addition to generating a green juice suitable for chromatography, both clarification methods have been demonstrated to yield similar reduction in infectious virus titer.

If necessary, the clarified green juice may also be subjected to a freeze and thaw as is indicated at S15 in FIG. 3. Specifically, the clarified green juice, clarified in either of steps S13 or S14, may be frozen, thawed and then re-centrifuged, as is indicated at step S16 in FIG. 3. The freezing and thawing causes precipitation of starchy material and additional contaminating plant proteins which are separated from the clarified green juice by a further centrifugation or filtration. This step S15 may optionally be performed depending upon the clarity of the green juice after filtration or centrifugation, and therefore aid in further downstream purification steps, but is not a required step of the present invention.

The volume of the clarified green juice is next normalized such that a plurality of samples containing diverse proteins can be simultaneously purified. During normalization, urea or glycerol may be added to predetermined concentrations and/or pH adjustment of the sample may occur. For example, urea at concentrations ranging from 50 mM to 4 M and glycerol at concentrations ranging from 5% w/v to 50% w/v may be employed and NaOH (sodium hydroxide) or a sodium phosphate or Tris buffer, may be used to raise pH from 7.2-7.3 to 7.5-8.0. It should be understood that during normalization, only pH adjustment may occur. Levels of urea and glycol may or may not be included depending upon the characteristics and properties of the desired protein of interest.

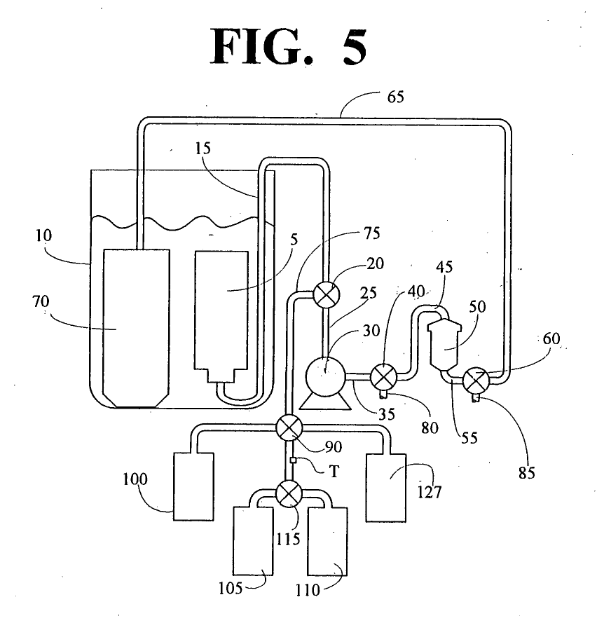

The normalized clarified green juices are then loaded into a purification apparatus, such as the apparatus described below with respect to FIGS. 5 through 11, as indicated at step S17 in FIG. 4.

Further description of the methods of the present invention depicted in FIG. 4 is now joined with a description of the apparatus depicted in FIGS. 5 through 11.

As shown in FIG. 5, the purification apparatus of the present invention includes a feed reservoir 5 that is initially filled with the clarified green juice and buffer solution, as indicated in the flowchart of FIG. 4 at step S17. During the purification process, the feed reservoir 5 is submerged in a larger receptacle 10 filled with a cooling agent, such as an ice water mixture in order to maintain the feed reservoir 5 and the clarified green juice at a temperature below 10° C., preferably at about 4° C. and more preferably as close to 0° C., but above the freezing point of the green juice. It is desirable to maintain the clarified green juice at a generally low temperature in order to minimize oxidation and proteolylic activity. It should be understood that any cooling agent, such as ice and water, may be used in the larger receptacle 10 in order to maintain the clarified green juice at a temperature above freezing, but below 10° C. Alternatively, a refrigeration mechanism may be employed to maintain a low temperature around the receptacle 5. Although not depicted, the feed reservoir 5, larger receptacle 10, and a flow-through collection reservoir 70 (described below) may be disposed within a robotic fluid handler in order to manipulate the fluids in a more automated fashion. Such fluid handlers include any of a variety of robotic fluid handling devices, such as those manufactured and sold by TECAN, Zurich Switzerland, including models such as the Genesis RSP, Robotic Sample Processor, Genesis Freedom, Modular Automated Workstation, or Genesis Workstation, Automated Workstation.

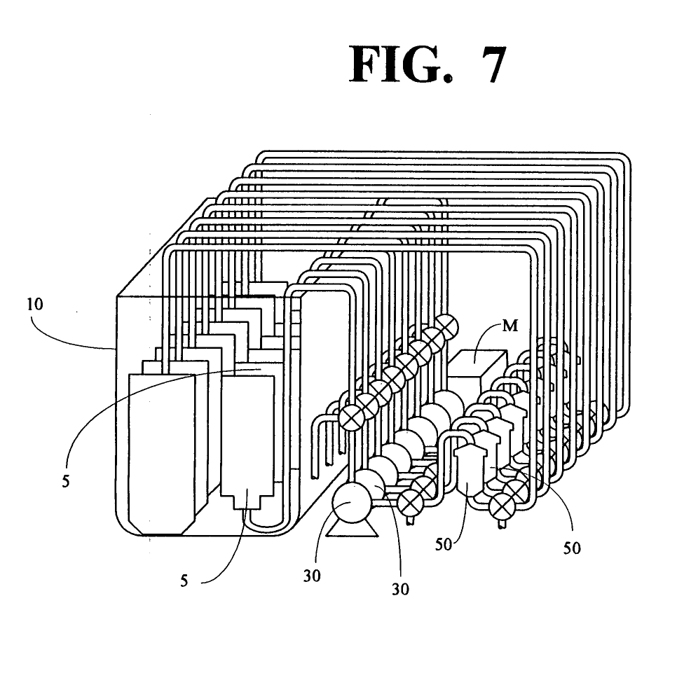

The feed reservoir 5 is connected to a tube 15 that is connected to a first valve 20. The first valve 20 is connected to a tube 25 that is further connected to a pump 30. The pump 30 may be any of a variety of pumps, but is preferably a low velocity pump that moves the clarified green juice through the purification apparatus of the present invention at a generally slow rate. For instance, the pump 30 may be a peristaltic pump such as a variable speed pump manufactured by ISMATEC® with a flow range of 0.01 to 44.4 mL/minute. Such pumps are also multi-channel pumps enabling simultaneous purification of multiple proteins, each in its own purification apparatus in a manner described in greater detail below with respect to FIG. 7.

The pump 30 is connected to a second valve 40, which is in turn connected to tube 45, which is connected to a column 50. The column 50 is connected to tubing 55 that is connected to a third valve 60. The third valve 60 is connected to a tube 65 that is connected to a flow-through collection reservoir 70. It should be understood from the following description that clarified green juice loaded into the feed reservoir 5 is transported via pumping action of the pump 30 from the feed reservoir 5, through the various tubes 15, 25, 35, 45, 55 and 65 and through the column 50 and valves 20, 40 and 60, into the collection reservoir 70.

The column 50 possesses a porous frit that retains a material therein, but allows the flow of fluid therethrough such that there can be contact and potential interaction between the flowing fluid and the retained material. In the purification apparatus of the present invention, the material in the column 50 is, for instance, an affinity resin, such as those marketed by Qiagen®, or other similar material for temporarily retaining the desired protein of interest. As the clarified green juice flows through the column 50 the protein of interest is attracted to and retained on the affinity resin.

The valves 20, 40 and 60 are connected to tubes 75, 80 and 85, respectively and are included in the purification apparatus for a variety of purposes. In purification mode, the valve 20 is set to allow fluid communication (fluid flow) from the tube 15 to the tube 25. The valve 20 may also be set to allow fluid flow from the tube 75 into the tube 25 for cleaning purposes, for removal of the purified protein of interest (as is described further below), or for priming the pump 30 and system equilibration, among other functions. The valve 20 may also be set to allow fluid communication between the tube 15 and 75.

In purification mode, the valve 40 is set to allow fluid communication between the tube 35 and the tube 45. However, the valve 40 may be set to allow fluid flow between the tube 35 and the tube 80 for cleaning or priming the pump 30, or the valve 40 may be set to allow fluid flow between the tube 80 and the tube 45 for washing the column 50 or for removal of the purified protein of interest.

In purification mode, the valve 60 is typically set to allow fluid communication between the tube 55 and the tube 65. The valve 60 may also be set to allow fluid communication between the tube 55 and tube 85 to allow for washing of the column 50 or for removal of the isolated protein of interest in the column 50. The valve 60 may also be set to allow fluid communication between the tube 85 and 65 to permit flushing and cleaning of the tube 65.

It should be understood that the valve 40 is optional and may alternatively be omitted from the apparatus depicted in FIG. 5, depending upon the application of the apparatus.

Under some circumstances, the system may need to be primed. Specifically, fluid may be introduced from receptacle 100 to line 15, line 25, pump 30, line 35, line 45 and line 55 by manipulation of the valve 20 and 85. Typically, the system would be primed with the column 50 removed, and lines 45 and 55 directly connected to one another. After the system has been primed, the removable column 50 is re-inserted between lines 45 and 55, as shown in FIG. 5. In the priming process, the tube 15 is also filled with priming fluid. It should be understood that no green juice would be present in the reservoir 5 during priming and may be poured or delivered via automated fluid handler into the reservoir 5 after priming is complete. The lines 45 and 55 may include specific couplings (not shown) to allow easy removal and replacement of the column 50 during the priming process.

For operation of the purification system, clarified green juice is put into the juice receptacle 5. Thereafter, the pump 30 is operated to draw clarified green juice out of the juice receptacle 5, into the tube 15, through the valve 20 and of course the pump 30, through tubes 35 and 45 and valve 40 and into the column 50. In the column 50, the clarified green juice interacts with the material disposed in the column 50, and ideally, all protein of interested is retained within the column 50 while the remainder of the clarified green juice flows out of the column 50, basically as waste. The waste juice passes through the tubes 55 and 65 and valve 85 and into the collection reservoir 70.

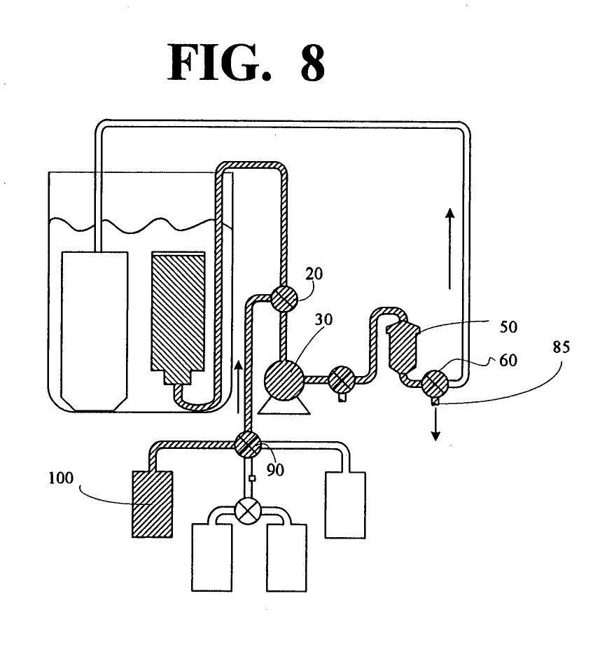

Returning now to FIG. 4, prior to purification, the affinity resin and column 50 must be conditioned prior to the beginning of the purification mode, as is indicated at S18 in FIG. 4 as indicated by the text Equilibrate Column. To equilibrate the column 50, an equilibration solution is provided in receptacle 100 that simulates the characteristics of the green juice and buffer solution in receptacle 5, as shown in FIG. 8. For instance, the equilibration solution typically has the same pH as the clarified green juice and buffer and further includes identical concentrations of urea, PEG and/or glycerol if present in the green juice and buffer solution. The equilibrate solution is pumped from the receptacle 100 through the column 50 and to waste via the tubing 85, as is indicated in FIG. 8.

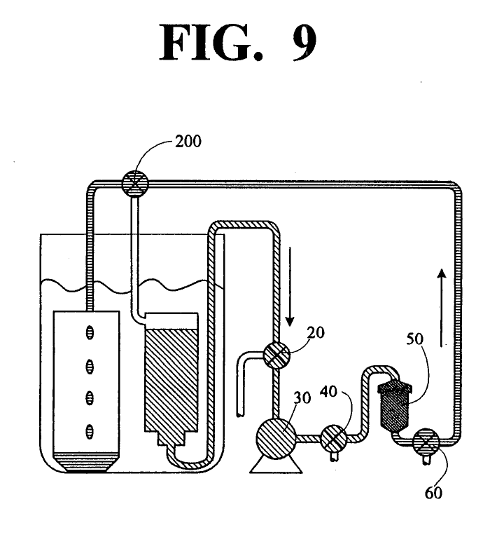

Thereafter, the valves 20 and 60 are set for purification mode and the clarified green juice and buffer solution mixture are pumped from the receptacle 5, through the column 50 and into the collection reservoir 70, as is depicted in FIG. 9 and indicated at S19 in FIG. 4. As described above, the affinity resin captures the protein of interest by interaction with the tag in the protein. The pump 30 pumps the clarified green juice and buffer solution mixture through the column 50 at a predetermined rate such that the residence time within the column 5 and hence, the affinity resin, is between 30 seconds and 5 minutes, but preferably, the pump 30 pumps at a rate that gives the green juice a residence time of approximately 1 minute within the column.

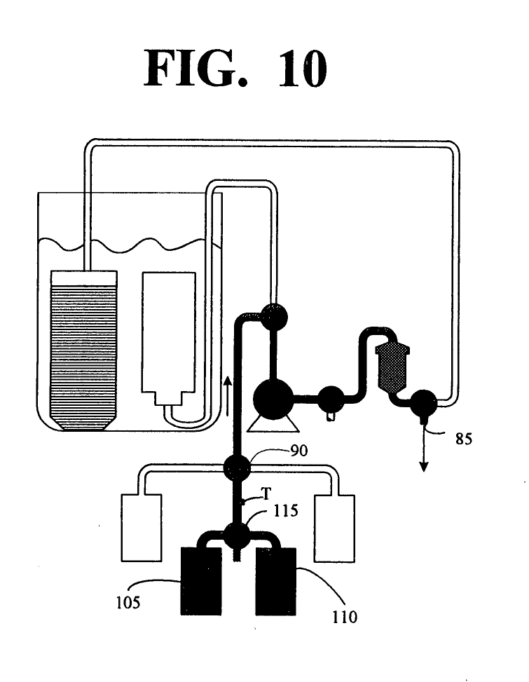

After all of the mixture has passed through the column 50, contaminates must be washed out and certain buffer components, e.g. PEG and urea removed, as indicated at step S20 in FIG. 4. As shown in FIG. 10, the contaminates are washed out of the column 50 by at least one of two solutions stored in receptacles 105 and 110 via control of a proportioning valve 115. For instance, for many proteins, a buffered solution in receptacle 110 containing low concentrations of the competitive inhibitor imidazole (10-90 mM) may be used to reduce contaminate protein interactions with the affinity resin. Alternatively, initially the solution in receptacle 110 contains a buffered solution with urea, glycerol and/or PEG concentration similar to the clarified green juice. This is passed through the column 50 and its flow gradually but linearly decreased as the flow from the receptacle 105 is linearly increased. The buffered solution in reservoir 105 contains different concentrations of urea and glycerol and/or PEG, typically zero. Therefore, the concentration of these unwanted components gradually decreases during this process in order to avoid rapid changes in the conditions within the column 50 which may negatively impact the retained tagged protein. As shown in FIG. 10, the wash exhausts via the tubing 85.

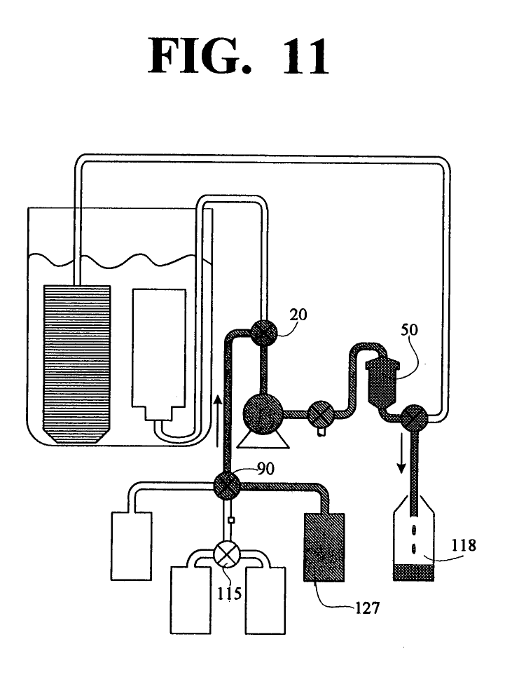

Next, as indicated in step S21 in FIG. 4, the column 50 is eluted to remove the protein of interest and fed into a reservoir, as shown in FIG. 11. Typically, a predetermined elution solution, such as phosphate buffered saline containing imidazole or EDTA at 100-200 mM, is provided in reservoir 127, shown in FIGS. 5 and 11. The solution in reservoir 127 is fed via valves 90 and 20 through the column 50 releasing the captured protein of interest from the affinity material disposed in the column 50 such that it is captured in reservoir 118, as shown in FIG. 11.

Alternatively, prior to elution from column 50, the protein of interest may be re-folded in situ on the column matrix through the introduction of a linear gradient of renaturation buffer (e.g. phosphate buffered saline, tris buffered saline or other buffers used in renaturation) after washing. For example, many histidine tagged proteins are purified under denaturing conditions, exposing the histidine tag at either the carboxy or amino terminus, thereby increasing binding of the tag to binding groups present on the metal affinity matrix. The histidine-tagged proteins are then subsequently eluted in their denatured state from the metal affinity matrix by lowering the pH of the buffer passing through the column or introducing a high concentration of imidazole or EDTA. The eluted proteins, especially at higher concentrations, sometimes fall, or precipitate, out of solution. This may be caused by intermolecular interactions between hydrophobic groups which are exposed due to the denatured state of the eluted protein. If the proteins cannot be resolubilized, the overall yield of protein is decreased. However, by the introduction of a linear gradient of renaturation buffer after washing, the protein may be allowed to re-fold while bound to the affinity matrix. Upon re-folding, the previously exposed hydrophobic groups are shielded, preventing intermolecular hydrophobic interactions and precipitation of the proteins.

It is important that a gradient is employed for inducing re-folding of the protein. Although practice of the claimed methods is not dependent upon an understanding of the mechanism of the invention, it is believed that the gradual introduction of renaturation buffer assists in the proper folding of the protein while bound to the affinity matrix, giving the bound protein time to properly re-fold into complex tertiary or quartenary structures. After the re-folding of the protein of interest on the column, the protein may now be eluted from the column with the introduction of elution buffer.

Linear gradient makers may be used where re-folding of the protein of interest in situ while bound to the affinity matrix is desired. Linear gradient makers allow the gradual introduction of the renaturation buffer over a set volume or period of time. Linear gradient makers may employ at least one pump or proportioning valve for drawing from two reservoirs containing the starting and final buffer, such as reservoirs 105 and 110 depicted in FIGS. 5 and 10. For example, a first reservoir may contain denaturation buffer and a second reservoir renaturation buffer. A regulating valve or proportioning valve 115 between the first reservoir and second reservoir regulates the inflow of the two buffers, thus changing the composition of the column running buffer from the second reservoir to the first reservoir. The composition of the column running buffer at the beginning of the run consists primarily of denaturation buffer. The buffer composition is then gradually changed, with the introduction of renaturation buffer from the second reservoir into the first reservoir until eventually the column running buffer comprises only renaturation buffer, allowing the gradual re-folding of the protein. Alternatively, a mixing chamber (not shown) may be employed whereby the contents of the first reservoir and second reservoir are pumped into the mixing chamber for passing onto the column. Like above, the relative ratios of the first and second reservoir vary, with the composition of the running buffer consisting of primarily denaturation buffer at the beginning of the run, and primarily renaturation buffer at the end of the run. Upon refolding of the protein, an elution buffer may be passed over to remove the tagged protein from the affinity matrix.

Gradual introduction of the renaturation buffer may occur by stepwise, instead of a linear gradient, introduction of renaturation buffer. For example, buffer solutions of decreasing salt or urea concentrations may be flowed over the column in a stepwise fashion. It is appreciated that one of ordinary skill in the art will appreciate the many ways by which a gradual introduction of renaturation buffer may take place to re-fold a denatured protein of interest in situ on the affinity matrix.

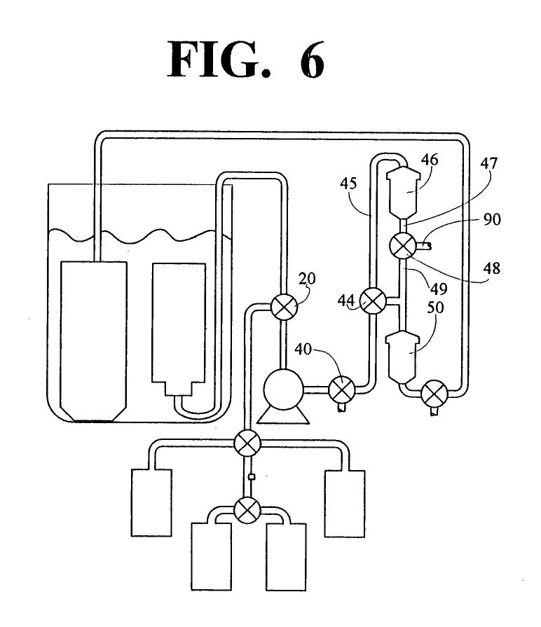

There are groups of proteins that are difficult to separate from one another. Therefore, in an alternate embodiment depicted in FIG. 6, a sacrificial column 46 may alternatively be added to the apparatus depicted in FIG. 5. Specifically, in FIG. 6, the sacrificial column 46 is connected to the tube 45 and is in fluid communication with the tube 45 such that any juice flowing from the tube 45 flows into the column 46. The column 46 is further connected to tube 47 for fluid communication therewith. The tube 47 is connected to a valve 48, the valve 48 is connected to the tube 49, and the tube 49 is connected to the previously described column 50. Otherwise all elements of the system depicted in FIG. 6 are identical to the elements in embodiment depicted in FIG. 5.

The valve 48 is further connected to a tube 90. In purification mode, the valve 48 is set to direct flow of juice from the tube 47 to the tube 49 and into the column 50. However, the valve 48 may further be set to allow fluid communication between the tube 47 and tube 90. As well the valve 48 may be set to allow fluid communication between the tube 90 and tube 49 for cleaning purposes, flushing purposes or for removal of purified protein in a manner described in greater detail below.

Further, as shown in FIG. 9, the apparatus may be provided with a recycling valve 200 in order to provide the green juice with multiple passes through the column 50.

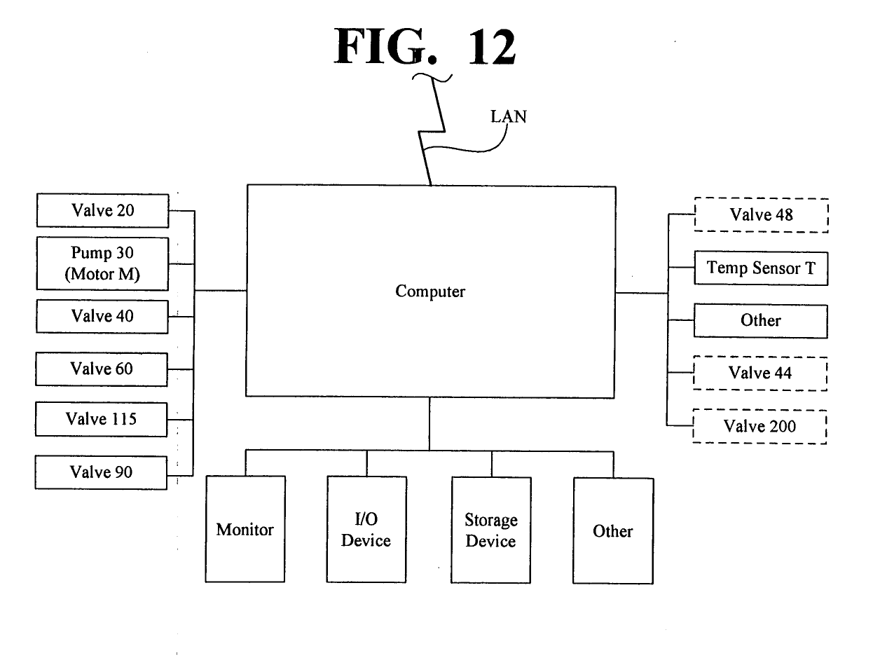

As shown in FIG. 12, a computer is provided for automated control of each of the embodiments of the apparatus of the present invention depicted in FIGS. 5 thru 11 and described above. Specifically the computer is connected to the pump and various valves in the apparatus. It should be understood that the above description of the operation of the systems depicted in FIGS. 5, and 8-11 is also applicable to the apparatus in FIG. 6 and the apparatus in FIG. 7.

The apparatus in FIG. 7 depicts a system wherein a plurality of flow channels separate from one another, each having its own column 50, each operating in parallel for simultaneous purification of a plurality of proteins. Specifically, a single peristaltic pump motor M coupled to each of the pumps 30 provides pumping action of the multiple flow channels such that green juice may flow through the plurality of columns simultaneously. Further, each of the feed reservoirs 5 are submersed in a single ice bath 10. The peristaltic pump motor operates to give the desired column residence time, as mentioned above, such that the green juice flows through the columns 50 at a rate to ensure reliable capture of the protein of interest from green juice. Since the flow rate may be slow, it may take a significant amount of time for acceptable purification of the protein of interest. If only one flow channel, such as the flow channel of the apparatus depicted in FIG. 5 is employed, purification of multiple proteins takes a prohibitive amount of time. Therefore, putting a plurality of flow channels together in a single apparatus for parallel, simultaneous extraction of a plurality of proteins provides a significant advantage in the protein purification process.

The computer depicted in FIG. 12 is connected to the motor M of the pump 30 or in the alternative, a single pump 30, and valves 20, 40, 60, 90 and 115. The computer may further be connected to temperature sensor T and pressure sensors (not shown) for control of the multiple channel system. Pressure sensors may alternatively be provided on the apparatus at locations upstream and downstream of the column 50 in order to control the fluid flow therethrough. Further, for the alternative embodiments depicted in FIGS. 6 and 9, the valves 44, 48 and 200 may also be connected to the computer for control thereof.

The computer depicted in FIG. 12 is a part of the LIMS (laboratory information management system) that is depicted in the block diagram of FIG. 1 and is also connected via a LAN (local area network) to the server depicted in FIG. 2. As is indicated in FIG. 1, the LIMS is an integral part of the processes of the present invention and includes software for tracking all biological material, such as gene sequences, the DNA sequences used to express proteins, the proteins expressed, the production levels of each protein, the expression system used to produce those proteins, all data relating to the pre-screening process, correlations to searched database information and all of the various steps carried out for producing and purifying the proteins of interest. The LIMS includes the computer system depicted in FIG. 2 and the computer depicted in FIG. 12. The computer depicted in FIG. 12 further includes programming enabling it to control the various valves 20, 40, 60, 90 and 115, and optional valves 44, 48 and 200 in order to isolate and elute the protein of interest as described above.

In accordance with the present invention, it is possible to purify quantities of proteins measured in milligrams in a cost effective and efficient manner. Other methods and apparatus allow for extremely large quantities (measured in 100 g to Kg) or extremely small quantities (measured in μs), therefore the methods and apparatuses of the present invention fulfill a need.

### EXAMPLE 1

### Expression and Purification of a Plurality of Proteins for Antibody Production

A subset of proteins, with emphasis on markers whose expression is restricted to either the lung or the brain, were selected for GENEWARE®-based expression and subsequent purification in parallel. Protein databases such as the Human Protein Index (HPI) and SWISS-PROT were screened for potential proteins and a subset chosen based on the availability of full-length clones from both in-house and commercially available gene collections. Each full-length clone was assigned a sequence ID (SeqID) to permit tracking of the DNA sequence and resulting protein in the laboratory information management system (LIMS), from vector generation through to confirmation (FIG. 1). Using the polymerase chain reaction (PCR), with primers complementary to each DNA sequence, the open reading frame of each target was amplified, subsequently purified and ligated into the appropriate GENEWARE® expression vector. The vector was modified to contain a histidine tag sequence such that expressed proteins would possess a tag at the N-terminus. The resulting vectors were screened to confirm insert integrity and orientation. Successful cloning events were evaluated for protein expression while those that failed were reintroduced into the cloning workflow.

For screening, sufficient in-vitro transcript was generated for each clone to inoculate three 21-day old Nicotiana benthamiana plants. Twelve to fourteen days after inoculation, the plant material above the inoculated leaves was harvested, weighed and macerated to obtain a green juice. In a deep-well block (96 well), one volume green juice was combined with 2 volumes extraction buffer (25 mM Tris pH 8.0, 500 mM NaCl, 2 mM PMSF, 7 mM β-mercaptoethanol) and adjusted to 4% w/v PEG (1500 ul final volume), to simulate the extract obtained during protein production. After storage for half an hour at 4° C., the green juice was centrifuged at 3000×G for 20 minutes to obtain a clarified green juice, containing the target protein. To capture the target protein, 700 ul of the clarified green juice was combined with 25-ul affinity resin (Qiagen Ni-NTA) in a 96-well filter plate and incubated for one hour at room temperature. The filter was sufficiently hydrophobic to retain the clarified green juice, which could be removed following incubation, by centrifugation at 1000×G for 5 minutes. The affinity resin, with the captured protein, was retained by the filter and washed twice with 700 ul wash buffer (16 mM Tris, pH 8.0, 330 mM NaCl, 5 mM imidazole), with centrifugation at 1000 G for 5 minutes between washes. Recovery of the target protein from the affinity resin was achieved by incubating the resin in 60 ul elution buffer (16 mM Tris, pH 8.0, 150 mM NaCl, containing either 200 mM Imidazole or 200 mM EDTA) for 5 minutes and centrifuging (1000×G for 5 minutes) to recover the eluant. The elution step was repeated to yield 120 ul of final product. To assess the expression level of each tagged protein, the eluent from each purification was analyzed by SDS-PAGE. If a protein band of approximately the correct molecular weight (+/−20%) was observed following Coomassie staining, and no co-migrating bands were observed in the negative controls, successful expression of target protein was assumed. The protein level was quantified by densitometry, using a bovine serum albumin standard. This variable was inputted into the LIMS system, together with the recorded plant mass and the number of plants required to produce the target protein was determined.

For protein production, N benthamiana plants were sown in lots of nine. To facilitate tracking and inoculation, the number of plants required for each protein target was rounded up to the nearest multiple of nine. The expression level for each protein will vary greatly and subsequently so too will the number of plants required to achieve a given protein level. Lots varying from nine to ninety-six 21-day old plants were typically used and the in vitro transcription reactions scaled accordingly. Twelve to fourteen days after inoculation, the plant material above the inoculated leaves was harvested, weighed and combined with two volumes of chilled extraction buffer. The extraction buffer was vacuum infiltrated into the plant material to ensure even buffer/plant material distribution and the green juice obtained using a commercial juice extractor. PEG was added to 4% w/v and the green juice stored at 4° C. for half and hour, to permit aggregation and precipitation of the chlorophyll-containing component of the extract. The green juice was clarified by filtration, employing 4% w/v perlite as a filtration aid. The clarified green juice was adjusted to 10% v/v glycerol, to minimize hydrophobic protein interactions with the affinity resin and the extract volumes normalized with extraction buffer. Each channel of the pre-equilibrated purification apparatus was loaded with clarified green juice containing a particular target protein. In the case where the volume of green juice for a given target protein was substantially greater than for the other target proteins, the clarified green juice was divided into two of more of the channels and the purified proteins pooled following elution from the affinity resin. The clarified green juice was passed over the affinity resin and the histidine-tagged protein retained on the Ni-NTA affinity resin. Contaminating plant proteins were removed by passing 10 column volumes of wash buffer over the column and the target protein recovered using an elution buffer containing 200 mM EDTA. The composition of the extraction buffer, wash buffer and elution buffer were identical to those employed in the screening step. Aliquots of each eluant were analyzed by SDS-PAGE and densitometry of the Coomassie-stained protein bands performed, to determine the concentration of the protein. Where necessary the proteins were concentrated by ultrafiltration and all proteins were dialyzed into phosphate buffered saline, prior to storage at −20° C. SDS-PAGE was performed on the final concentrated and dialyzed proteins, to determine protein purity and tryptic MALDI was performed to confirm protein identity.

Table 1 summarizes the results for production runs were between 5 and 15 unique proteins were expressed using GENEWARE® and purified in parallel. Based on the screening, sufficient plants were inoculated to obtain 1.5 mg of purified protein, with a minimum of 9 plants per target protein. In production mode the required protein level was achieved or exceeded for 10 of the 27 targets. In the case of 11 targets a second round of production with appropriately adjusted plant numbers would be performed to meet the protein requirement. For the six targets were no protein was recovered, GENEWARE® expression on a 9-plant lot would be performed to confirm the result. If no protein is recovered following this purification, the SeqIDs are identified as incompatible with GENEWARE® and evaluated in another expression system e.g. mammalian.

TABLE 1

| Squence | | | | | aa | size | Total |
|---|---|---|---|---|---|---|---|
| ID | Target origin | Swissprot | Protein name | Tissue | length | Da | mg |
| 1231035 | M000000TUF | P08263 | Glutathione S-transferase A1 | Liver | 221 | 25500 | 11.9 |
| 1230610 | Cardiovascular | P15090 | Fatty acid-binding protein, adipocyte | Urinary bladder | 131 | 14588 | 8.1 |
| | genome unit cDNA | | | | | | |
| | array | | | | | | |
| 1231070 | M000000FNR | Q06520 | ALCOHOL SULFOTRANSFERASE | Liver | 285 | 33648 | 7.0 |
| | | | (EC 2.8.2.2) (HYDROXYSTEROID) | | | | |
| 1232042 | Cardiovascular | P29373 | Retinoic acid-binding protein II, | Skin | 137 | 15562 | 4.7 |
| | genome unit cDNA | | cellular | | | | |
| | array | | | | | | |
| 1231073 | M000000FLD | P21695 | GLYCEROL-3-PHOSPHATE | Liver | 349 | 37462 | 3.3 |
| | | | DEHYDROGENASE [NAD+], | | | | |
| | | | CYTOPLASMIC (EC 1.1. | | | | |
| 1230553 | LSB Swissprot | Q15126 | PMVK_HUMAN | Liver | 192 | 21864 | 3.0 |
| | List 1 | | PHOSPHOMEVALONATE | | | | |
| | | | KINASE (PMKASE) | | | | |
| 1230669 | Pfizer rat/human | P40616 | ADP-ribosylation factor-like protein 1 | Umbilical vein | 181 | 20417 | 2.9 |
| | tox targets | | | endothelial cells | | | |
| 1230617 | HPI List 1 | P07226 | TROPOMYOSIN, FIBROBLAST | Fibroblast | 248 | 28522 | 2.7 |
| | | | NON-MUSCLE TYPE (TM30-PL) | | | | |
| 1231042 | M000001HYD | P05388 | 60S acidic ribosomal protein P0 | Brain & Muscle | 317 | 34273 | 1.9 |
| 1230630 | Pfizer rat/human | P08865 | 40S ribosomal protein SA, aka Colon | Lung, Brain, Muscle, | 295 | 32854 | 1.5 |
| | tox targets | | carcinoma laminin-binding protein | Placenta, Urinary | | | |
| | | | | bladder & Uterus | | | |
| 1231080 | M000000FNB | P49419 | ANTIQUITIN | Kidney, Liver & | 511 | 55366 | 1.0 |
| | | | | Placenta | | | |
| 1232088 | HPI List 2 | P40121 | Macrophage capping protein | Placenta | 348 | 38517 | 0.9 |
| 1231036 | M000000TUX | Q14749 | Glycine N-methyltransferase | Liver & Placenta | 294 | 32611 | 0.9 |
| 1232027 | LSB Swissprot | P50550 | UBCI_HUMAN UBIQUITIN- | Fetal brain | 158 | 18007 | 0.6 |
| | List 1 | | CONJUGATING ENZYME E2-18 | | | | |
| | | | KDA (UBIQUITIN-PRO | | | | |
| 1232007 | Pfizer rat/human | P32119 | Peroxiredoxin 2, aka Thioredoxin | Brain | 198 | 21892 | 0.4 |
| | tox targets | | peroxidase 1 | | | | |
| 1232035 | Cardiovascular | P07195 | L-lactate dehydrogenase B chain | T-cell & Muscle | 333 | 36507 | 0.4 |
| | genome unit cDNA | | | | | | |
| | array | | | | | | |
| 1231089 | M000000FMV | P17516 | 3-alpha-hydroxysteroid | Liver | 323 | 37095 | 0.4 |
| | | | dehydrogenase | | | | |
| 1232074 | Pfizer rat/human | P09417 | Dihydropteridine reductase | N/A | 244 | 25803 | 0.3 |
| | tox targets | | | | | | |
| 1230569 | Cardiovascular | P14174 | HUMAN MACROPHAGE | Liver | 115 | 12345 | 0.2 |
| | genome unit cDNA | | MIGRATION INHIBITORY | | | | |
| | array | | FACTOR (MIF) | | | | |
| 1232056 | Cardiovascular | Q01543 | Friend leukemia integration 1 | Bone marrow | 452 | 50982 | 0.2 |
| | genome unit cDNA | | transcription factor | | | | |
| | array | | | | | | |
| 1231056 | M000000FN3 | P32754 | ALCOHOL SULFOTRANSFERASE | Liver | 392 | 44803 | 0.1 |
| | | | (EC 2.8.2.2) (HYDROXYSTEROID) | | | | |
| 1230660 | Cardiovascular | Q99685 | LYSOPHOSPHOLIPASE | Lung & brain | 313 | 34292 | 0.0 |
| | genome unit cDNA | | HOMOLOG | | | | |
| | array | | | | | | |
| 1230623 | Cardiovascular | P10451 | Osteopontin precursor | Liver, Kidney & | 314 | 35422 | 0.0 |
| | genome unit cDNA | | | Brain | | | |
| | array | | | | | | |
| 1230652 | Pfizer rat/human | P23821 | 40S ribosomal protein S7 | N/A | 194 | 22127 | 0.0 |
| | tox targets | | | | | | |
| 1232067 | Cardiovascular | Q16217 | Argininosuccinate synthetase protein | N/A | 11 | 1024 | 0.0 |
| | genome unit cDNA | | [Fragment] | | | | |
| | array | | | | | | |
| 1231028 | M000001HYT | P56211 | cAMP-regulated phosphoprotein 19 | Brain | 111 | 12192 | 0.0 |
| 1231061 | M000001HZD | P30084 | Enoyl-CoA hydratase, mitochondrial | Liver | 290 | 31371 | 0.0 |
| | | | [Precursor] | | | | |

Various details of the invention may be changed without departing from its or its scope. Furthermore, the foregoing description of the embodiments according resent invention is provided for the purpose of illustration only, and not for the e of limiting the invention as defined by the appended claims and their equivalents.

1-15. (canceled)

16. An apparatus for purification of a plurality of biological substances, comprising:
a first reservoir having a first solution including a biological substance disposed therein;
a second reservoir having a second solution including a biological substance disposed therein;
a first valve, said first reservoir being connected to said first valve;
a second valve, said second reservoir being connected to said second valve;
a first column connected for fluid communication to said first valve directing the first solution through said first column, said first column having a biological substance retaining material disposed therein;
a second column connected for fluid communication to said second valve directing the second solution through said second column, said second column having a biological substance retaining material disposed therein; and
a computer connected to said first and second valves for automated control of said apparatus;
wherein flow paths through said first and second columns are separated from one another and flow through said first and second columns may be effected simultaneously.

17. An apparatus for purification of a plurality of biological substances as set forth in claim 16, further comprising:
a third valve downstream from said first column for controlling flow of fluid out of said first column;
a fourth valve downstream from said second column for controlling flow of fluid out of said second column; and
wherein said third and fourth valves are connected to said computer such that said computer controls operation of said third and fourth valves.

18. An apparatus for purification of a plurality of biological substances as set forth in claim 17, further comprising:
a first pump upstream of said first valve;
a second pump upstream of said second valve; and
wherein said first and second pumps are connected to said computer such that said computer controls operation of said first and second pumps.

19. An apparatus for purification of a plurality of biological substances as set forth in claim 18, wherein said first and second pumps are connected to a single motor, operation of said motor being controlled by said computer.

20. An apparatus for purification of a plurality of biological substances as set forth in claim 18,
wherein said first reservoir, said first valve, said first column, said third valve downstream from said first column, and said first pump all define a first flow path;
said second reservoir, said second valve, said second column, said fourth valve downstream from said second column, and said second pump all define a second flow path separate and distinct from said first flow path; and
said apparatus further comprises a third flow path in parallel with and separate and distinct from said first and second flow paths for purification of another biological substance, said third flow path comprising:
a third reservoir having a third solution including a biological substance disposed therein;
a third column having a biological substance retaining material disposed therein;
a third pump;
a fifth valve connected between said third pump and said third column; and
a sixth valve downstream from said third column for controlling flow of fluid out of said third column.

21. The apparatus of claim 18, wherein each of said pumps comprises a peristaltic pump.

22. The apparatus of claim 21, wherein each of said peristaltic pumps comprises a variable speed pump.

23. The apparatus of claim 22, wherein each of said variable speed pumps is operable within a range of from about 0.01 to about 44.4 mL/min.

24. The apparatus of claim 16, further comprising a cooling system for maintaining a temperature of said first and second reservoirs at a value above 0° C. and not in excess of 10° C.

25. The apparatus of claim 18, further comprising:
a first buffer solution reservoir system including a buffer solution disposed therein;
a second buffer solution reservoir system including a buffer solution disposed therein;
a fifth valve connected between said first buffer solution reservoir system and an inlet of said first pump, for controlling flow of buffer solution to said first column;
a sixth valve connected between said second buffer solution reservoir system and an inlet of second pump, for controlling flow of buffer solution to said second column; and
wherein said fifth and sixth valves are connected to said computer such that said computer controls operation of said fifth and sixth valves.

26. The apparatus of claim 25, wherein:
said first buffer solution reservoir system comprises two buffer solution reservoirs and a blending valve for allowing flow from either or both of the two buffer solution reservoirs to said fifth valve, said blending valve being connected to said computer such that said computer controls operation of said blending valve.

27. The apparatus of claim 18, further comprising:
a first elution reservoir including an elution solution disposed therein;
a second elution reservoir including an elution solution disposed therein;
a fifth valve connected between said first elution reservoir and an inlet of said first pump, for controlling flow of elution solution to said first column;
a sixth valve connected between said second elution reservoir and an inlet of said second pump, for controlling flow of elution solution to said second column; and
wherein said fifth and sixth valves are connected to said computer such that said computer controls operation of said fifth and sixth valves.
